(19) United States
(12) Patent Application Publication
(10) Pub. No.: US 2025/0339518 A1
(43) Pub. Date: Nov. 6, 2025

(54) DUAL STING/RIG-I AGONIST ONCOLYTIC MINICELLS AS IN SITU IMMUNIZATION AGENTS AND METHODS OF USE

(71) Applicant: Vaxiion Therapeutics, LLC, San Diego, CA (US)

(72) Inventor: Matthew J. Giacalone, San Diego, CA (US)

(21) Appl. No.: 19/264,504

(22) Filed: Jul. 9, 2025

Related U.S. Application Data
(63) Continuation of application No. PCT/US2024/011105, filed on Jan. 10, 2024.
(60) Provisional application No. 63/479,442, filed on Jan. 11, 2023.

(51) Int. Cl. A61K 39/39; A61K 31/7084; A61K 35/744; A61K 39/00; A61K 47/69; A61P 35/00

(52) U.S. Cl. CPC A61K 39/39; A61K 31/7084; A61K 35/744; A61K 47/6901; A61P 35/00; A61K 2039/55594; A61K 2039/575; A61K 2039/585; A61K 2039/80

(57) ABSTRACT

Disclosed herein are compositions including oncolytic bacterial rBMCs, and the use of the composition as in situ immunization agents against cancer. In some embodiments, the rBMCs are endowed with the ability to simultaneously stimulate upstream intracellular mediators of a cytokine response.

The present application is a continuation of PCT Patent Application No. PCT/US2024/011105, filed Jan. 10, 2024, which claims the benefit of priority of U.S. Provisional Application No. 63/479,442, filed Jan. 11, 2023, each of which is incorporated by reference in its entirety herein.

### BACKGROUND

### Field

The present application is drawn to compositions and methods for the production, purification, formulation, and use of dual STING/RIG-I agonist oncolytic eubacterial minicells for use as in situ immunization agents.

### Description of the Related Art

The following description of the background is provided to aid in understanding the disclosure, but is not admitted to describe or constitute prior art to the disclosure provided herein. The contents of the articles, patents, and patent applications, and all other documents and electronically available information mentioned or cited in this application, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other documents.

In situ immunization agents represent an emerging treatment paradigm for cancer therapy. The therapeutic premise of this approach is to expose tumors while they are still in place (e.g. “in situ”) to an immune-stimulatory agent to break peripheral immune tolerance to promote and/or restore the immune system's ability to recognize and effectively attack tumors. There are two critical components required to mount a successful response. The first is the availability of tumor antigens for cross-presentation by dendritic cells to initiate an antitumor lymphocyte response. The second is the production of Type I interferons (IFN). Several treatment modalities have been investigated for this purpose, yet none yet has unlocked the full potential of this approach. The primary reason being that these agents can only stimulate one component of this response and not the other. The approach that has shown the most promise is the use of oncolytic virus(es). These agents promote a strong Type I IFN response upon infection, and then go on to complete their lifecycle, the terminal event for which is cell lysis (oncolysis), which promotes release of tumor antigens. However, this presents at least two problems with respect to the intended purpose. First, the replication of every oncolytic virus developed to date is negatively impacted by the production of Type I IFN (an innate antiviral immune response). Not surprisingly, it is well recognized that oncolytic viruses do not work well in tumors that have intact Type I IFN pathways. Second, the Type I IFN response is an early antiviral response that peaks within 24 hours of infection. Replication of the virus and completion of the viral lifecycle (viral burst), which is required to promote tumor antigen availability, does not occur until 24 to 72 hours later depending on the virus type employed. Thus, the peak of the Type I IFN response and the peak of tumor antigen availability are asynchronous and suboptimal.

Other approaches, including some utilizing bacterial minicells have been described, but like oncolytic viruses, these approaches also suffer from suboptimal and asynchronous timing of antigen availability and innate immune stimulation via the Type I IFN pathway. In addition, these previous attempts to utilize bacterial minicells to deliver agonists of cytosolic nucleic acid receptors such as Stimulator of Interferon Genes (STING) and retinoic acid inducible gene (RIG-I) in an attempt to stimulate Type I IFN production fall short because they do not include, envision, or conceive of utilizing an endosomal escape mechanism to allow the agonist(s) to traverse the endosome into the cytosol where STING and RIG-I molecules, the molecular targets of these agonists, reside.

Based on the observed limitations of these approaches it is evident that there is room for improvement with respect to developing improved in situ immunization agents that can better synchronize the peak Type I IFN response with the peak availability of tumor antigens. The present disclosure describes novel use of rapid tumor targeted oncolytic recombinant bacterial minicells (rBMCs) endowed with the ability to simultaneously stimulate upstream intracellular mediators of the Type I IFN response, including the STING and RIG-I cytosolic nucleic acid sensing pathways as a new class of in situ immunization agents for use in the treatment of cancer.

### SUMMARY

Described herein are compositions and methods for treating, inhibiting, or ameliorating cancer.

Accordingly, some embodiments provided herein relate to recombinant bacterial minicells (rBMCs) for the inhibition or treatment of cancer. In some embodiments, the rBMCs include a surface localized targeting molecule; a cytolysin protein; and an agonist of an intracellular mediator of a Type I IFN response. In some embodiments, the rBMCs include invasin; perfringolysin O (PFO); and a stimulator of interferon genes (STING) agonist or a retinoic acid inducible gene I (RIG-I) agonist. In some embodiments, the surface localized targeting molecule includes invasin. In some embodiments, the cytolysin protein includes perfringolysin O (PFO). In some embodiments, the agonist is a STING agonist, a RIG-I agonist, or both. In some embodiments, the STING agonist includes c-di-GMP, 2′3′cGAMP, 3′3′cGAMP, cAIMP, cAIMP difluoro, cAIMP (PS) 2 Difluor (Rp/Sp), a cyclic di-nucleotide, ADU-S100, MK-1454, BMS-986301, E7766, GSK3745417, SB 11285, or TAK-676, or any combination thereof. In some embodiments, the RIG-I agonist includes an uncapped 5′triphosphate RNA. In some embodiments, the 5′triphosphate RNA ranges from about 30 to about 2,000 nucleotides in length. In some embodiments, the 5′triphosphate RNA is single stranded or double stranded. In some embodiments, the RIG-I agonist includes polyI: C, SLR14, polyU/UC, RN7SL1, M8, 3p-siBCL2, MK-4621, and BO-112. In some embodiments, the rBMC expresses one or more recombinant tumor selective antigens. In some embodiments, the one or more recombinant tumor selective antigens is HER-2, K-RAS, H-RAS, N-RAS, MAGE, c-MYC, MUC-1, PSMA, CEA, ETA, CA-125, p53, AFP, Tyrosinase, oncofetal protein, or antigens produced by oncogenic viruses. In some embodiments, the rBMC is produced from a naturally invasive strain of bacteria. In some embodiments, the naturally invasive strain of bacteria includes Salmonella spp., Listeria spp., Mycobacterium spp., Shigella spp., Yersinia spp., or Escherichia coli. In some embodiments, the rBMC is an oncolytic rBMC.

Some embodiments provided herein relate to compositions that include any of the rBMCs provided herein. Some embodiments provided herein relate to pharmaceutical compositions. In some embodiments, the pharmaceutical compositions include any rBMC as described herein and a pharmaceutically acceptable carrier. In some embodiments, the compositions further include an immune checkpoint inhibitor. In some embodiments, the immune checkpoint inhibitor includes an inhibitor against PD-1, PD-L1, PD-L2, PD-L3, PD-L4, CTLA-4, LAG-3, IDO, B7-H3, B7-H4, GITR, 4-1BB, OX40, CD27, KIR2DL, CSF1R, CD40L, KIR, or TIM-3.

Some embodiments provided herein relate to methods of inhibiting or treating cancer. In some embodiments, the methods include administering to a subject having cancer a composition that includes any of the rBMCs described herein. In some embodiments, the composition is a pharmaceutical composition. In some embodiments the methods include administering to a subject having cancer a pharmaceutical composition including a targeted oncolytic recombinant bacterial minicell (rBMC). In some embodiments, the bacterial rBMC is configured to stimulate upstream intracellular mediators of a Type I IFN response. In some embodiments, the intracellular mediators of the Type I IFN response include stimulator of interferon genes (STING) and retinoic acid inducible gene I (RIG-I) nucleic acid sensing pathways.

In some embodiments, the method inhibits the growth of cancer. In some embodiments, the method inhibits or delays the onset of cancer. In some embodiments, the cancer is a solid tumor, a metastatic tumor, or a liquid tumor. In some embodiments, the cancer is epithelial, fibroblast, muscle, or bone origin. In some embodiments, the cancer is adenocarcinoma, sarcoma, fibrosarcoma, eye, brain, bone, breast, lung, pancreatic, prostatic, testicular, ovarian, gastric, intestinal, mouth, tongue, pharynx, hepatic, anal, rectal, colonic, esophageal, urinary bladder, gall bladder, skin, uterine, vaginal, penal, renal cancer, non-Hodgkin's lymphoma, myeloma, Hodgkin's lymphoma, acute lymphocytic leukemia, chronic lymphocytic leukemia, acute myeloid leukemia, or chronic myeloid leukemia.

In some embodiments, the methods further include administering an immune checkpoint inhibitor therapy. In some embodiments, the immune checkpoint inhibitor therapy includes administration of one or more immune checkpoint inhibitors. In some embodiments, the one or more immune checkpoint inhibitors includes an inhibitor against PD-1, PD-L1, PD-L2, PD-L3, PD-L4, CTLA-4, LAG-3, IDO, B7-H3, B7-H4, GITR, TIGIT, 4-1BB, OX40, CD27, KIR2DL, CSF1R, CD40L, KIR, or TIM-3. In some embodiments, the rBMC includes a surface localized targeting molecule. In some embodiments, the surface localized targeting molecule includes invasin. In some embodiments, the rBMC includes a cytolysin protein. In some embodiments, the cytolysin protein is perfringolysin O (PFO).

In some embodiments, the rBMC includes a STING agonist, a RIG-I agonist, or both. In some embodiments, the STING agonist includes c-di-GMP, 2′3′cGAMP, 3′3′cGAMP, cAIMP, cAIMP difluoro, cAIMP (PS) 2 Difluor (Rp/Sp), a cyclic di-nucleotide, ADU-S100, MK-1454, BMS-986301, E7766, GSK3745417, SB 11285, or TAK-676, or any combination thereof. In some embodiments, the RIG-I agonist includes an uncapped 5′triphosphate RNA. In some embodiments, the 5′triphosphate RNA ranges from about 30 to about 2,000 nucleotides in length. In some embodiments, the 5′triphosphate RNA is single stranded or double stranded. In some embodiments, the RIG-I agonist includes polyI: C, SLR14, polyU/UC, RN7SL1, M8, 3p-siBCL2, MK-4621, and BO-112. In some embodiments, the rBMC expresses one or more recombinant tumor selective antigens. In some embodiments, the one or more recombinant tumor selective antigens is HER-2, K-RAS, H-RAS, N-RAS, MAGE, c-MYC, MUC-1, PSMA, CEA, ETA, CA-125, p53, AFP, Tyrosinase, oncofetal protein, or antigens produced by oncogenic viruses. In some embodiments, the rBMC is produced from a naturally invasive strain of bacteria. In some embodiments, the naturally invasive strain of bacteria includes Salmonella spp., Listeria spp., Mycobacterium spp., Shigella spp., Yersinia spp., or Escherichia coli.

Some embodiments provided herein relate to use of the compositions provided herein for treating, inhibiting, or ameliorating a cancer.

### BRIEF DESCRIPTION OF THE DRAWINGS

FIGS. 1A-1D depict murine MB49 urothelial carcinoma tumor cell lines upregulate programmed death ligand 1 (PD-L1) (FIG. 1A) and major histocompatibility complex I (MHC-I) (FIG. 1B) as determined by flow cytometry in a PFO dependent manner. PD-L1 and MHC-I upregulation (FIG. 1C) is dependent upon Type I IFN as indicated by a loss of upregulation when naïve MB49 cells have been preincubated with a receptor function blocking antibody against IFNAR1 (the sole receptor for Type I IFN) before transfer of treated supernatants. IFN-β (detected by ELISA) is present in supernatants of treated MB49 cells but not those of VAX-I treated cells, indicating a need for PFO-mediated endosomal escape to elicit Type I IFN production (FIG. 1D).

FIGS. 2A-2B depict activation of sting and RIG-I using embodiments of the compositions described herein.

FIGS. 3A-3C depict that genetic ablation of STING confirms activation of the STING pathway and RIG-I pathway using embodiments of the compositions described herein.

FIGS. 4A-4C depict that genetic ablation of RIG-I confirms activation of the RIG-I pathway using embodiments of the compositions described herein.

FIG. 5 depicts genetic ablation of both STING and RIG-1, confirming activation of both the STING pathway and RIG-I pathway using embodiments of the compositions described herein.

FIGS. 6A-6C depict tumor growth rates and survival of STING and RIG-I positive tumors using embodiments of the compositions described herein.

FIG. 7 depicts antitumor activity of embodiments of the compositions described herein is T-cell dependent and leads to tumor specific antitumor immunologic memory.

FIGS. 8A-8D depict in vivo induction of Type I IFN production after intratumoral administration of embodiments of the compositions described herein.

FIGS. 9A-9B depict that embodiments of the compositions described herein are more potent when loaded with exogenous ADU-S100 than free ADU-S100.

FIG. 10 depicts activation of Type I IFN in human tumor cell lines in a PFO-dependent manner using embodiments of the compositions described herein.

FIG. 11 depicts a diagram highlighting endosome escape.

FIG. 12 depicts a diagram of dual STING/RIG-I agonistic rBMC.

FIG. 13 depicts a diagram of an antibody targeted rBMC.

### DETAILED DESCRIPTION

Although the disclosure is described in various exemplary alternatives and implementations as provided herein, it should be understood that the various features, aspects, and functionality described in one or more of the individual alternatives are not limited in their applicability to the particular alternative with which they are described. Instead, they can be applied alone or in various combinations to one or more of the other alternatives of the embodiments described herein, whether the alternatives are described or whether the features are presented as being a part of the described alternative. The breadth and scope of the present disclosure should not be limited by any exemplary alternatives described or shown herein.

Disclosed herein are compositions and methods for treating cancer. Embodiments of the compositions include, for example, oncolytic recombinant bacterial minicells (rBMCs) having the ability to simultaneously stimulate intracellular mediators of a cytokine response. Embodiments of the methods and uses include administering the compositions described herein to a subject having or suspected of having cancer.

### Definitions

As used herein, the term “rBMC(s)” refers to achromosomal bacterial minicell(s) and is synonymous with the terms “recombinant bacterial minicell(s)”, “bacterial minicell(s)”, “eubacterial minicells”, and “minicells”.

As used herein, the term “recombinant invasive immunomodulatory rBMC” refers to an rBMC that has been genetically engineered to express and display heterologous rBMC surface proteins capable of stimulating internalization, for example by endocytosis, into eukaryotic cells to deliver immunogenic, immunotherapeutic, or other immune stimulatory molecules.

As used herein, the term “naturally invasive immunomodulatory rBMC” refers to an rBMC produced from a normally invasive bacterium such that said rBMCs express and display naturally occurring rBMC surface proteins capable of stimulating internalization into eukaryotic cells.

As used herein, the term “immunotherapy” refers to the use of an immunomodulatory compound, including, for example an immunomodulatory rBMC, to generate an innate immune response that has beneficial effect with respect to the elimination or slowing the progression of disease, especially cancer.

As used herein, the term “adherent rBMC” refers to a rBMC that is capable of binding and adhering to the surface of a non-constitutively phagocytic eukaryotic cell without stimulating appreciable endocytosis of said rBMCs.

As used herein, the term “muco-adherent rBMC” refers to a rBMC that is capable of binding and adhering to a mucosal surface.

As used herein, the term “oncolytic rBMC” refers to a rBMC that is capable of stimulating tumor cell lysis.

As used herein, the term “integrin targeted rBMCs” refers to rBMCs that express and display the pan-Betal-integrin-targeting cell surface molecule Invasin from Yersinia pseudotuberculosis or any functional equivalents thereof. Integrin targeted rBMCs are also defined as those rBMCs that include a surface-localized integrin-specific antibody or antibody derivative.

As used herein, the term “dual STING/RIG-I rBMCs” refers to rBMCs including an endosomal escape protein, one or more STING and/or RIG-I agonists, wherein the endosomal escape protein is a preformed polypeptide that is further capable of facilitating tumor cell lysis before, during, or shortly after agonizing intracellular STING and/or RIG-I pathways to stimulate Type I IFN.

As used herein, the term “dual STING/RIG-I agonist VAX014 rBMCs” refers to rBMCs that express and display the pan-Betal-integrin-targeting cell surface molecule Invasin from Yersinia pseudotuberculosis and any functional equivalents thereof wherein the rBMCs include perfringolysin O (PFO) to facilitate endosomal escape followed by oncolysis and can agonize intracellular nucleic acid sensing pathways, including but not limited to STING and/or RIG-I, to stimulate Type I IFN before, during, or shortly after stimulating oncolysis.

As used herein, the term “formulated VAX014 drug product”, refers to a sterile formulation of dual STING/RIG-I agonist oncolytic VAX014 rBMCs wherein one or more excipients is included. Formulated VAX014 drug product may be formulated as a freeze-dried lyophile or as a suspension.

As used herein, the term “prokaryotic cell division gene” refers to a gene that encodes a gene product that participates in the prokaryotic cell division process. Many cell division genes have been discovered and characterized in the art. Examples of cell division genes include, but are not limited to, zipA, sulA, secA, dicA, dicB, dicC, dicF, ftsA, ftsI, ftsN, ftsK, ftsL, ftsQ, ftsW, ftsZ, minC, minD, minE, seqA, ccdB, sfiC, and ddlB.

As used herein, the term “transgene” refers to a gene or genetic material that has been transferred naturally or by any of a number of genetic engineering techniques from one organism to another. In some embodiments, the transgene is a segment of DNA containing a gene sequence that has been isolated from one organism and is introduced into a different organism. This non-native segment of DNA may retain the ability to produce RNA or protein in the transgenic organism, or it may alter the normal function of the transgenic organism's genetic code. In some embodiments, the transgene is an artificially constructed DNA sequence, regardless of whether it contains a gene coding sequence, which is introduced into an organism in which the transgene was previously not found.

As used herein, an agent is said to have been “purified” if its concentration is increased, and/or the concentration of one or more undesirable contaminants is decreased, in a composition relative to the composition from which the agent has been purified. In some embodiments, purification includes enrichment of an agent in a composition and/or isolation of an agent therefrom.

The term “sufficiently devoid of parental cells”, synonymous with “sufficiently devoid”, as used herein refers to a composition of purified rBMCs that have a parental cell contamination level that has little or no effect on the toxicity profile and/or therapeutic effect of targeted therapeutic rBMCs.

The term “domain” or “protein domain” used herein refers to a region of a molecule or structure that shares common physical and/or chemical features. Non-limiting examples of protein domains include hydrophobic transmembrane or peripheral membrane binding regions, globular enzymatic or receptor regions, protein-protein interaction domains, and/or nucleic acid binding domains.

The terms “eubacteria” and “prokaryote” are used herein as these terms are used by those in the art. The terms “eubacterial” and “prokaryotic” used herein encompass eubacteria, including both Gram-negative and Gram-positive bacteria, prokaryotic viruses (e.g., bacteriophage), and obligate intracellular parasites (e.g., Richettsia, Chlamydia, etc.).

The term “oncolytic polypeptide” is synonymous with “oncolytic protein”, “tumorlytic polypeptide”, and “tumorlytic protein” and the terms are used interchangeably herein to refer to any collection of diverse protein molecule types that have a lytic effect when introduced into a eukaryotic organism or cell (e.g., a mammal such as human). An oncolytic polypeptide can be a cholesterol-dependent cytolysin, a phospholipase, a functional enzyme, a cell-penetrating peptide, a perforin, or any combination and/or plurality of the proceeding. In some cases, the oncolytic polypeptide also facilitates endosomal escape before, during, or shortly after initiating oncolysis.

The term “endosomal escape agent” is synonymous with “endosomal escape agent” and “endosomal disrupting polypeptide” and the terms are used interchangeably herein to refer to any collection of diverse protein molecule types capable of disrupting endosomal membranes to facilitate cytosolic delivery of endosomal contents following internalization of bacterial rBMCs into endosomes when internalized into a eukaryotic organism or cell (e.g., a human cell). An endosomal escape agent can be a cholesterol-dependent cytolysin, a phospholipase, a functional enzyme, a cell-penetrating peptide, a perforin, or any combination and/or plurality of the proceeding. An endosomal escape protein facilitates the delivery of other molecules, including agonists of cytosolic nucleic acid receptors including but not limited to STING and RIG-I agonists, as well as other small molecule drugs, nucleic acids, polypeptides, lipids, and bioactive agents from the endosomal compartment into the cytosol of a mammalian cell. An endosomal escape protein may also impart oncolytic activity, but such activity is not a requirement of endosomal escape protein(s).

The terms “immunogen” and “antigen” are interchangeable and used herein to refer to polypeptides, carbohydrates, lipids, nucleic acids, and other molecules to which an antigen-specific antibody, cellular, and/or allergenic response may be mounted against.

The term “tumor specific antigen(s)” is used herein to refer to tumor selective polypeptides, carbohydrates, lipids, nucleic acids, and other molecules to which an antigen-specific antibody, cellular, and/or allergenic response may be mounted against. Tumor specific antigens arise from one or more genetic mutations, including but not limited to missense mutations, frameshift mutations, nonsense mutations, point mutations, and the like.

The term “overexpression” used herein refers to the expression of a functional nucleic acid, polypeptide or protein encoded by DNA in a host cell, wherein the nucleic acid, polypeptide or protein is either not normally present in the host cell, or wherein the nucleic acid, polypeptide or protein is present in the host cell at a higher level than that normally expressed from the endogenous gene encoding the nucleic acid, polypeptide or protein.

The term “modulate” as used herein means to interact with a target either directly or indirectly to alter the activity of the target to regulate a biological process. The mode of “modulate” includes, but is not limited to, enhancing the activity of the target, inhibiting the activity of the target, limiting the activity of the target, and extending the activity of the target.

The term “heterologous” as used herein refers to a protein, gene, nucleic acid, imaging agent, buffer component, or any other biologically active or inactive material that is not naturally found in a rBMC or rBMC-producing bacterial strain that is expressed, transcribed, translated, amplified or otherwise generated by rBMC-producing bacterial strains that harbor recombinant genetic material coding for said heterologous material or coding for genes that are capable of producing said heterologous material (e.g., a bioactive metabolite not native to the parent cell).

The term “foreign nucleic acid” as used herein refers to a nucleic acid not naturally found in a eukaryotic cell. Examples include bacterial and viral nucleic acids.

The term “foreign di-cyclic nucleotide” as used herein refers to a di-cyclic nucleotide not naturally found in a eukaryotic cell. Examples include bacterial di-cyclic nucleotides such as di-cGMP and di-cAMP as well as synthetic di-cyclic nucleotides and di-cyclic nucleotide analogs that activate one or more isoforms of human STING (e.g., those not existing in nature but developed by chemistry).

The term “STING agonist” as used herein refers to a small molecule drug(s), nucleic acid(s), di-cyclic nucleotide(s), or synthetic analog(s) thereof, or peptide(s) that activates one or more isoforms of human STING. Activation of STING can result in downstream signaling of the Tank-binding kinase 1 (TBK-1) pathway and/or the canonical NF-kappa B pathway and/or the noncanonical NF-kappa B pathway.

The term “RIG-I agonist” as used herein refers to a small molecule drug(s), nucleic acid(s), di-cyclic nucleotide(s), or synthetic analog(s) thereof, or peptide(s) that activates RIG-I to produce Type I IFN. Activation of RIG-I can result in downstream signaling of the Tank-binding kinase 1 (TBK-1) pathway and/or the canonical NF-kappa B pathway and/or the noncanonical NF-kappa B pathway.

The term “cytosolic nucleic acid receptor” as used herein refers to a diverse family of mammalian cytosolic proteins capable of recognizing cytosolic nucleic acids including but not limited single stranded RNA, double stranded RNA, double stranded DNA, single stranded DNA, hybridized DNA: RNA molecules, and synthetic derivatives of each and any of these nucleic acid species. Cytosolic nucleic acid receptors include but are not limited to cGAS, RIG-I, MDA5, MAVS, Protein kinase R (PKR), AIM2, IFI16, and nucleotide oligomerization domain (NOD) like receptor (NLR) family members.

The term “exogenous” as used herein refers to a protein (including antibodies), gene, nucleic acid, small molecule drug, di-cyclic nucleotide, imaging agent, buffer, radionuclide, or any other biologically active or inactive material that is not native to a cell, or in the case of a rBMC, not native to the parent cell of the rBMC. Exogenous material differs from heterologous material by virtue of being generated, purified, and added separately.

The term “therapeutic” as used herein means having a biological effect or combination of biological effects that prevents, inhibits, eliminates, or prevents progression of a disease or other aberrant biological processes in an animal.

The term “diagnostic” as used herein means having the ability to detect, monitor, follow, and/or identify a disease or condition in an animal (including humans) or from a biological sample including but not limited to blood, urine, saliva, sweat and fecal matters.

The term “theranostic” as used herein means having the combined effects of a therapeutic and a diagnostic composition.

The term “recombinantly expressed” as used herein means the expression of one or more nucleic acid(s) and/or protein(s) from a nucleic acid molecule that is artificially constructed using modern genetic engineering techniques wherein the artificially constructed nucleic acid molecule does not occur naturally in rBMCs and/or rBMC-producing bacterial strains wherein the artificial nucleic acid molecule is present as an episomal nucleic acid molecule or as part of the rBMC-producing bacterial chromosome.

The term “episomal” as used herein means a nucleic acid molecule that is independent of the chromosome(s) of a given organism or cell.

The term “detoxified” as used herein refers to a modification made to a composition or component thereof that results in a significant reduction in acute toxicity to the modified composition or component thereof, regardless of what the causative biological basis for toxicity to the composition or component thereof happens to be.

As used herein, the term “bioactive molecule” refers to a molecule having a biological effect on a eukaryotic organism or cell (e.g., a mammal such as human) when introduced into the human organism or cell. Bioactive molecules include, but are not limited to, therapeutic nucleic acids, therapeutic polypeptides (including protein toxins), and therapeutic small molecule drugs.

### Description

The present disclosure relates to the use of bacterial rBMCs as in situ immunization agents designed to more optimally stimulate the immune system within tumors in such a way as to generate a systemic antitumor immune response. The rBMCs described herein include but are not limited to fast-acting tumor-targeted dual STING/RIG-I agonistic oncolytic rBMCs.

As described herein, the present disclosure utilizes recombinant bacterial minicells (rBMCs) designed to address the limitations of previous approaches by including a critically enabling endosomal escape protein that also serves to facilitate tumor lysis (oncolysis). Embodiments of the present disclosure are directed to solving the suboptimal and asynchronous timing of tumor antigen availability and Type I IFN production. The rapid acting oncolytic rBMCs described herein are designed to first transfer STING and/or RIG-I agonist(s) into the cytosol of the target cell such that the said agonist can reach its cytosolic target(s), STING and or RIG-I, to stimulate Type I IFN production during or in close approximation to the time in which tumor cell lysis (oncolysis) occurs. As described in various embodiments herein, the absence of an endosomal escape mechanism results in minimal Type I IFN induction, whereas incorporation of such a mechanism results in robust and rapid Type I IFN both in vitro and in vivo. The incorporation of an endosomal escape protein as well as the presence of STING and RIG-I in tumors is shown herein to be critical for antitumor activity as well the development of protective antitumor immunologic memory, which is the ultimate therapeutic objective of in situ immunization. Also described herein are novel compositions and methods for rBMCs comprising encapsulated endogenous and/or exogenous STING and/or RIG-I agonists along with an endosomal escape protein for purposes of targeted delivery of these agonists to the cytosol with the intent of driving Type I IFN production during or in close approximation to oncolysis. As demonstrated herein, this unique combination of features produces a more robust Type I IFN response, the peak of which overlaps with the peak of oncolysis.

As described herein, exogenous STING and/or RIG-I agonists may be incorporated into tumor-targeted bacterial rBMCs along with a critically enabling endosomal escape protein that also serves to facilitate oncolysis. Including an endosomal escape protein facilitates transfer of the STING and/or RIG-I agonist into the cytosol of the target cell such that the said agonist can reach its cytosolic target(s), STING and or RIG-I. Previous attempts to utilize bacterial rBMCs to deliver agonists of cytosolic nucleic acid receptors capable of stimulating Type I IFN production fall short because they do not envision, include, or conceive of utilizing an endosomal escape protein to allow the agonist(s) to traverse the endosome into the cytosol where the molecular target of the agonist resides. The importance and impact of doing so is included herein.

The recombinant fast-acting tumor-targeted dual STING/RIG-I agonistic oncolytic rBMCs described herein are designed to cure the deficiencies of previous approaches by incorporating an endosomal escape protein. As demonstrated herein, this unique combination of features produces a much higher Type I IFN response, the peak of which overlaps with the peak of oncolysis. rBMCs used in the context of the present disclosure may be combined with immune checkpoint inhibitor therapies to further enhance and/or maintain the antitumor response to in situ immunization with said rBMCs. The combination of rBMC-mediated tumor antigen exposure and adjuvant properties associated with optimized STING and/or RIG-I activation synergize with immune checkpoint inhibitors to improve anti-tumor immune responses, including but not limited to T-cell and NK-cell mediated cytotoxic lymphocyte (CTL) responses against tumors. The effectiveness of this combination therapy can be enhanced by further combination with other treatment modalities known to aid in tumor antigen availability, immune cell activation, epigenetic modulation, anti-metabolite activity, and proteasome inhibition.

rBMCs are achromosomal, membrane-encapsulated biological nanoparticles (approximately 250-500 nm in diameter) that are formed by bacteria following a disruption in the normal division apparatus of bacterial cells. In essence, rBMCs are small, metabolically active replicas of normal bacterial cells with the exception that they contain no chromosomal DNA and as such, are non-dividing, non-viable, and non-infectious. Although rBMCs do not contain bacterial chromosomes, plasmid DNA molecules (smaller than chromosomes), RNA molecules (of all subtypes and structures), di-cyclic nucleotides, native and/or recombinantly expressed proteins, and other metabolites have all been shown to segregate into rBMCs. rBMCs are uniquely suited as in vivo delivery vehicles because they can be engineered to combine one or more different naturally occurring, heterologous, or exogenous molecular components into a single particle where each component is present in discreet amounts. This is in stark contrast to live bacterial-based delivery vehicles where live bacteria are capable of division and persistence, while generating unknown quantities of molecular components de novo after administration in vivo. Persistence and propagation of living bacterial delivery vehicles can lead to many different complications including infection, organ failure, sepsis, and death. In short, rBMCs can be “engineered” to preferentially encapsulate, be coupled to, or absorb biologically active molecules, including various nucleic acids, proteins, small molecule drugs, and any combination thereof for subsequent generation of biological responses in both prophylactic and therapeutic medicinal applications where the prevention, maintenance, and/or inhibition of disease by way of said biologic response is desirable.

Genetically engineered bacterial rBMCs have been used directly as anti-cancer agents as described in U.S. Pat. No. 7,183,105, which is incorporated herein by reference in its entirety. For example, it has been taught within U.S. Pat. No. 7,183,105 that rBMCs can be engineered to use rBMC surface-localized antibodies to target and deliver small molecule drugs, peptides, proteins, and various nucleic acids, together or in concert directly to cancer cells to exert a direct targeted anticancer effect. Other investigators have also reported the same findings as those taught in U.S. Pat. No. 7,183,105, with respect to the use of rBMCs as targeted delivery vehicles, as illustrated in U.S. Ser. Nos. 11/211,098, 10/588,028, and 10/581,990, each of which is incorporated herein by way of reference. On the other hand, U.S. Pat. No. 9,267,108 teach that rBMCs can be engineered and utilized as anti-cancer therapies capable of exerting indirect and non-selective anti-tumor effects. Each reference teaches the same approach to using rBMCs to specifically target and deliver anti-cancer agents only directly to tumor cells in vivo. The references included above do not teach the use of bacterial rBMCs to cause tumor-specific immune activation in response to treatment with rBMCs or oncolytic rBMCs or antibody targeted rBMC. An antibody targeted rBMC refers to an rBMC having an antibody presented on the surface thereof, and which targets a receptor, as described herein. To the contrary, bacterial rBMC-based compositions designed to generate tumor-specific adaptive immune responses are described in U.S. Pat. Nos. 7,183,105, 7,396,822, and U.S. Ser. No. 13/397,313 and each only in the context of using the bacterial rBMC as a carrier of a recombinant tumor selective antigen (e.g., a tumor selective single antigen “cancer vaccine”). In other related art, provided in each of U.S. Ser. Nos. 11/211,098, 10/588,028, and 10/581,990, it is demonstrated that targeting, using an antibody selective for a known tumor selective cell surface receptor coupled to the surface of the rBMC vehicle is required for anti-tumor activity. Further, these references also indicate that when non-targeted rBMCs are used, no significant anti-tumor response is observed. In other related work, MacDiarmid and colleagues demonstrate that both non-targeted rBMCs and tumor-targeted rBMCs containing no cytotoxic drug payload, are equally incapable of generating an anti-tumor response and that both a targeting antibody and the cytotoxic payload are required (MacDiarmid, et al. Cancer Cell, 2007, Volume 11, p. 431-445). Additionally, the authors of this work claim therein, the benefits of evading the immune system, describe this desired feature as part of their rationale for design, and therefore explicitly teach away from using rBMCs as immunomodulatory therapeutics. None of the prior art references describe combination of bacterial rBMCs and oncolytic rBMCs with immune checkpoint inhibitors.

As previously summarized, other related art such as U.S. Ser. Nos. 15/725,008 and 16/725,008 describe the use of bacterial minicells loaded with various immune stimulants, including those that may stimulate Type I IFN production used in combination with other rBMCs that are loaded with a chemotherapeutic drug. This approach is suboptimal in at least two key ways. First, no endosomal escape mechanism is employed, which as described in the examples herein, is a critically enabling feature in terms of optimizing cytosolic delivery of a STING and/or RIG-I agonist. These agonists have poor membrane permeability properties and are subject to rapid degradation after prolonged retention in the harsh endosomal environment. For example, RIG-I agonists are typically 5′ppp-RNA molecules ranging in length from about 30 to about 2,000 nucleotides in length and do not freely traverse phospholipid bilayers such as those of endosomal vesicles. Similarly, STING agonists such as di-cyclic nucleotides typically exhibit poor cell permeability properties because their net electronegative charge is repelled by the net electronegative charge of mammalian cell plasma membranes. Second, antitumor activity in vivo relies on the further utilization and coadministration of a chemotherapeutic drug packaged within the same or different rBMCs. This leads to suboptimal and asynchronous timing of Type I IFN production (which is already minimal due to lack of endosomal escape) and the availability of tumor antigens (which is also minimal due to a lack of oncolysis). The chemotherapeutic drug(s) employed have been reported to induce immunogenic responses through the damage-associated molecular pattern (DAMP) response, but a lack of true oncolytic activity of these agents limits their ability to provide ample tumor antigens for cross-priming and the development of a robust protective antitumor immunologic response. Moreover, the mediocre immune responses that are mounted after administration of chemotherapy take time to manifest, thereby creating a scenario where the minimal Type I IFN production occurs much sooner than when the peak damage to tumor cells occurs.

Embodiments provided herein are novel and distinguished from previous teachings by virtue of incorporating a critically enabling endosomal escape mechanism to facilitate cytosolic delivery of STING and/or RIG-I agonists while facilitating rapid oncolysis of targeted tumor cells. The related art does not envision the use of oncolysis to facilitate tumor antigen release at the same time as STING and/or RIG-I agonist mediated Type I IFN production nor does it envision, hypothesize, or in any way consider or disclose the importance of including an endosomal escape agent to facilitate cytosolic delivery of STING and or RIG-I agonists.

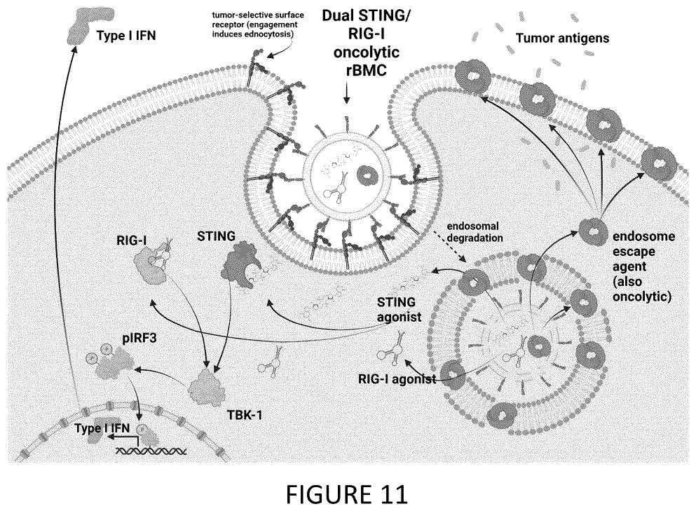

In contrast to the prior art, preferred embodiments of the present disclosure are specifically drawn to the use of bacterial rBMCs and dual STING/RIG-I agonist oncolytic bacterial rBMCs as improved in situ immunization agents by incorporating a critically enabling endosomal escape agent to facilitate transfer of STING and RIG/I agonists into the cytosol of targeted cells. One preferred embodiment provided herein is an oncolytic bacterial rBMC that serves as a targeted therapeutic capable of eliciting potent antitumor effects by simultaneous induction of tumor selective killing, tumor antigen exposure resulting from rBMC-mediated tumor cell lysis, and production of high levels of Type I IFN (FIG. 11). Thus, preferred embodiments described herein relate to rBMCs capable of killing tumor cells via a direct and rapid oncolytic mechanism (including for example, by way of targeted delivery of a pre-formed lytic polypeptide), whereby said polypeptide simultaneously facilitates both oncolysis and delivery of STING and or RIG-I agonist(s) to the cytosol of cells to promote Type I IFN production. Agonists of other cytosolic nucleic acid sensing molecules may also be incorporated into the disclosure (e.g. MAVS, MDA5, cGAS, LGP2, IFI16, and/or other members of the RLR family of cytosolic pattern recognition receptors) as may the addition of cell surface and/or endosomal pattern recognition receptor agonists (e.g., TLR agonists).

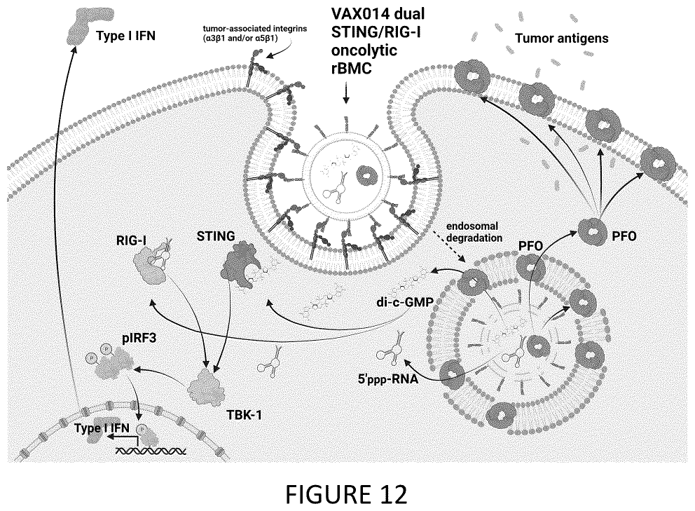

Some embodiments described herein relate to a dual STING/RIG-I agonistic VAX014 rBMC (FIG. 12). This composition is embodied by an rBMC comprising the surface localized pan-tumor integrin targeting molecule “invasin”, further comprising the cytolysin protein, perfringolysin O (PFO), further comprising a STING agonist, and further comprising a RIG-I agonist wherein the STING agonist is c-di-GMP, c-di-AMP, 2′3′cGAMP, 3′3′cGAMP, cAIMP, cAIMP difluoro, cAIMP (PS) 2 Difluor (Rp/Sp), and/or ADU-S100 and wherein the RIG-I agonist is an uncapped 5′triphosphate RNA. Variations of this composition are also preferred embodiments of the described compositions and include a composition embodied by an rBMC comprising the surface localized pan-tumor integrin targeting molecule “invasin”, further comprising the cytolysin protein, perfringolysin O (PFO), further comprising a STING agonist, and further comprising a RIG-I agonist wherein the STING agonist is MK-1454, BMS-986301, E7766, GSK3745417, SB 11285, or TAK-676 and wherein the RIG-I agonist is an uncapped 5′triphosphate RNA.

Some embodiments provided herein relate to a dual STING/RIG-I agonistic antibody-directed rBMC (FIG. 12). This composition is embodied by an rBMC comprising a surface localized antibody or antibody fragment targeting a tumor selective surface molecule, further comprising the cytolysin protein perfringolysin O (PFO), further comprising a STING agonist, and further comprising a RIG-I agonist wherein the STING agonist is c-di-GMP, c-di-AMP, 2′3′cGAMP, 3′3′cGAMP, cAIMP, cAIMP difluoro, cAIMP (PS) 2 Difluor (Rp/Sp), and/or ADU-S100 and wherein the RIG-I agonist is an uncapped 5′triphosphate RNA. Variations of this composition are described herein and include a composition embodied by an rBMC comprising a surface localized antibody or antibody fragment targeting a tumor selective surface molecule, further comprising the cytolysin protein, perfringolysin O (PFO), further comprising a STING agonist, and further comprising a RIG-I agonist wherein the STING agonist is MK-1454, BMS-986301, E7766, GSK3745417, SB 11285, or TAK-676 and wherein the RIG-I agonist is an uncapped 5′triphosphate RNA.

STING agonists that may be incorporated into embodiments described herein include bacterial cyclic dinucleotides (c-di-GMP and/or c-di-AMP), the eukaryotic cyclic dinucleotide 2′3′cGAMP, other cyclic di-nucleotides, 2′3′cGAMP, 3′3′cGAMP, cAIMP, cAIMP difluoro, cAIMP (PS) 2 Difluor (Rp/Sp), and any functional analog, small molecule, peptide, or nucleic acid mimetics of the preceding. Specifically, STING agonists include ADU-S100, MK-1454, BMS-986301, E7766, GSK3745417, SB 11285, and TAK-676.

RIG-I agonists that may be incorporated into embodiments described herein and include uncapped 5′triphosphate RNA molecules ranging in length from about 30 to about 2,000 nucleotides in length, for example 30, 40, 50, 60, 70, 80, 90, 100, 150, 200, 250, 300, 350, 400, 450, 500, 600, 700, 800, 900, 1000, 1100, 1200, 1300, 1400, 1500, 1600, 1700, 1800, 1900, or 2000, or an amount within a range defined by any two of the aforementioned values. These include both single stranded or double stranded duplex RNA molecules, as well as those that form short hairpin or other tertiary structures capable of being recognized by RIG-I. Specifically, RIG-I agonists include polyI: C, SLR14, polyU/UC, RN7SL1, M8, 3p-siBCL2, MK-4621, and BO-112. Other RIG-I agonists that may be recombinantly expressed and present in rBMCs include bacterial tRNAs, mRNAs, or other shorthair pin forming RNAs.

In some embodiments, rBMCs and oncolytic rBMCs are engineered to further express one or more recombinant tumor selective antigens to bolster the overall amount and presentation of tumor selective antigens released in response to rBMC treatment. Recombinant tumor selective antigens utilized in some embodiments include, but are not limited to HER-2, K-RAS, H-RAS, N-RAS, MAGE, c-MYC, MUC-1, PSMA, CEA, ETA, CA-125, p53, AFP, Tyrosinase, and any of oncofetal proteins, antigens produced by oncogenic viruses, and products of mutated genes (e.g., tumor antigens). Further, some embodiments include the incorporation of patient derived and patient-specific tumor antigens, whereby a patient suffering from cancer has a unique genomic signature from which tumor antigen sequences may be derived via in silico technologies and algorithms known in the art and then recombinantly expressed as the tumor antigen component of the rBMC or oncolytic rBMC prior to re-introduction into the patient. rBMCs further include recombinantly expressed tumor antigens and patient-specific antigens, including but not limited to oncolytic rBMCs expressing the same, are used in combination with one or more immune checkpoint inhibitors. Said immune checkpoint inhibitors include but are not limited to those against PD-1, PD-L1, CTLA-4, LAG-3, IDO, GITR, 4-1BB, OX40, CD27, KIR2DL, CSFIR, CD40L, and TIM-3.

In some embodiments, rBMCs, oncolytic rBMCs, dual STING/RIG-I agonist oncolytic rBMCs, and any of the foregoing rBMCs further include recombinant tumor selective antigens and/or patient-specific tumor antigens are administered in combination with CAR-T or CAR-N autologous or allogenic immune cell therapies. Recombinant tumor selective antigens utilized in the context of some embodiments include but are not limited to HER-2, K-RAS, H-RAS, N-RAS, MAGE, c-MYC, MUC-1, PSMA, CEA, ETA, CA-125, p53, AFP, Tyrosinase, and any of oncofetal proteins, antigens produced by oncogenic viruses, and products of mutated genes (e.g., tumor antigens). The CAR-T or CAR-N therapy expresses one or more chimeric antigen receptors specific for said tumor selective antigen. Further, some embodiments include the incorporation of patient derived and patient-specific tumor antigens, whereby a patient suffering from cancer has a unique genomic signature from which tumor antigen sequences may be derived via in silico technologies and algorithms known in the art and then recombinantly expressed as the tumor antigen component of the rBMC or oncolytic rBMC prior to re-introduction into the patient followed by administration of CAR-T or CAR-N therapy specific for the same. rBMCs further include recombinantly expressed tumor antigens and patient-specific antigens, including but not limited to oncolytic rBMCs expressing the same and combined with CAR-T and CAR-N therapies, are used in further combination with one or more immune checkpoint inhibitors. Said immune checkpoint inhibitors include but are not limited to those against PD-1, PD-L1, CTLA-4, LAG-3, IDO, GITR, 4-1BB, OX40, CD27, KIR2DL, CSF1R, CD40L, and TIM-3.

In some embodiments, rBMCs and oncolytic rBMCs include but are not limited to those produced from naturally invasive strains of bacteria including but not limited to invasive strains of Salmonella spp., Listeria spp., Mycobacterium spp., Shigella spp., Yersinia spp., and Escherichia coli. These naturally invasive rBMCs and oncolytic rBMCs will display naturally occurring rBMC surface-localized ligands that are capable of stimulating internalization of rBMCs into eukaryotic cells. It should be taken into consideration that naturally invasive rBMCs do not exist in nature per se but rather are engineered from non-rBMC producing invasive strains of bacteria using one or more of the genetic approaches to generating rBMCs as described herein.

In some embodiments, rBMCs and oncolytic rBMCs include but are not limited to those produced from non-invasive strains of bacteria. Many non-invasive strains of bacteria are known to the skilled artisan and include but are not limited to non-invasive strains of Escherichia coli, Salmonella spp., Shigella spp., Lactobacillus spp., Pseudomonas spp., and the like.

rBMCs have distinct mechanisms and advantages with respect to loading of immunomodulatory polypeptides (e.g. cytokines, protein toxins, and cytolysins) and nucleic acids (e.g. double stranded RNA, hairpin RNA, double stranded linear DNA). For example, immunomodulatory rBMC-producing parental bacterial cells can be used to recombinantly express/produce one or more cytokines, protein toxins, and cytolysins prior to or at the same time that rBMCs are being produced. Recombinant polypeptides are expressed, segregate into, and are encapsulated by rBMCs, and then utilized to enhance, modulate, and/or stabilize tumor-specific immune responses and cytotoxic responses elicited by rBMCs in vivo.

In cases where polypeptide(s) are pre-formed by the parental cell by way of recombinant expression from a prokaryotic expression cassette (either chromosomal or episomal in location) and is then packaged inside of the rBMCs as an oncolytic protein and/or tumor selective antigen, the half-life of the polypeptide(s) within the rBMC is increased by use of rBMC producing bacterial strains harboring one or more deletions or other non-functional mutations in protease genes (e.g., the Lon protease of E. coli) responsible for proteolysis. In the absence of the protease(s), the protein toxin molecules accumulate to a higher level, increasing the potency of targeted rBMCs delivering the therapeutic polypeptide molecules. In the case of E. coli rBMC producing strains, mutation or deletions can be introduced into one or more of the lon, tonB, abgA, ampA, ampM, pepP, clpP, dcp, ddpX/vanX, elaD, frvX, gcp/b3064, hslV, hchA/b1967, hyaD, hybD, hycH, hycI, iadA, ldcA, ycbZ, pepD, pepE, pepQ, pepT, pmbA, pqqL, prlC, ptrB, sgcX, sprT, tldD, ycaL, yeaZ, yegQ, ygeY, yggG, yhbO, yibG, ydpF, degS, ftsH/hflB, glpG, hofD/hopD, lepB, lspA, pppA, sohB, spa, yaeL, yfbL, dacA, dacB, dacC, degP/htrA, degQ, iap, mepA, nlpC, pbpG, tsp, ptrA, teas, umuD, ydcP, ydgD, ydhO, yebA, yhbU, yhjJ, and nlpD genes.

In cases where nucleic acid(s) are pre-formed by the parental cell by way of recombinant expression from a prokaryotic expression cassette (either chromosomal or episomal in location) and then packaged inside of the rBMCs as an immunopotentiator, the half-life of the nucleic acid(s) within the rBMC is increased by use of immunomodulatory rBMC producing bacterial strains harboring one or more deletions or other non-functional mutations in nuclease genes (e.g., the rnc nuclease of E. coli) responsible for double stranded RNA degradation. In the absence of the nuclease(s), immunomodulatory nucleic acid molecules accumulate to a higher level, increasing the potency of immunomodulatory rBMCs harboring said immunomodulatory nucleic acid molecules.

rBMCs intended for use as therapeutic agents in humans should contain few or no viable contaminants, such as viable parental bacterial cells or adventitious microbes introduced during the production process. In some embodiments, rBMC-based biopharmaceuticals formulated for human use conform to sterility under U.S. Pharmacopeia <71>. Some embodiments described herein include methods of sterilizing rBMC preparations intended for use in humans by exposure to sterilizing doses of gamma irradiation.

To further maximize safety and limit toxicity, rBMCs and oncolytic rBMCs may be derived from rBMC-producing parental bacterial strains containing a deletion of the lpxM/msbB gene. Deletion of the lpxM gene results in the production of de-toxified lipopolysaccharide (LPS) molecules that are immune-attenuated and may be more suitable for parenteral use. The lpxM gene (also referred to as the msbB gene) functions to add a terminal myristolic acid group to the lipid A portion of the LPS molecule and removal of this group (by way of elimination of the lpxM gene) results in marked detoxification of LPS. Specifically, detoxification is characterized by a decrease in the production of pro-inflammatory cytokines in response to exposure to LPS. It should be noted that this modification does not teach away from the present disclosure, as cytokines are still made using the detoxified form of LPS. The detoxification controls only the levels of cytokines produced, making it possible to dampen the acute sepsis-like pro-inflammatory response while allowing efficacy to be achieved without overt toxicity. This deletion can be introduced into any functionally equivalent gene of any Gram-negative or Gram-positive rBMC-producing strain to achieve the same effect. The enhanced safety profile can reduce the risk of infection and potential for developing sepsis, decrease the possibility of genetic reversion through recombination events with other bacteria, and minimize the risk of insertion events in the host. From a regulatory and manufacturing perspective, it is also preferred that antibiotic resistance markers be eliminated from the bacterial chromosome of the rBMC-producing parental cell strain. The use of most antibiotic resistance gene markers in rBMC-producing strains of bacteria is undesirable in order to comply with regulatory requirements imposed by the U.S. Food and Drug Administration (FDA) for use in humans. The FDA will only tolerate the use of the kanamycin resistance gene marker for selection purposes for bacteria or bacterial production strains wherein the final product is intended for use in humans.

Some embodiments provide a method of making rBMCs, including culturing the appropriate rBMC-producing bacteria disclosed herein and substantially separating rBMCs from the rBMC-producing parent cells, thereby generating a composition including therapeutic rBMCs. In some embodiments, the method further includes inducing rBMC formation from a culture of rBMC-producing parent cells. In some embodiments, the method further includes inducing expression of the gene encoding the genetic suicide endonuclease. In some embodiments, rBMC formation is induced by the presence of one or more chemical compounds selected from isopropyl β-D-1-thiogalactopyranoside (IPTG), rhamnose, arabinose, xylose, fructose, melibiose, and tetracycline. In some embodiments, the expression of the gene encoding the genetic suicide endonuclease is induced by a change in temperature. In some embodiments, the method further includes purifying the rBMCs from the composition. In some embodiments, the rBMCs are substantially separated from the parent cells by a process selected from the group including but not limited to centrifugation, filtration, ultrafiltration, ultracentrifugation, density gradation, immunoaffinity, immunoprecipitation, and any combination of the preceding purification methods.

The present disclosure describes the novel use of eubacterial rBMCs and oncolytic eubacterial rBMCs for purposes of stimulating the immune system in such a way as to have potent and specific anti-tumor effects mediated, in full or in part, by an adaptive immune response, including, for example, a tumor-specific CTL response, enhanced by exposure of tumor to said rBMCs in vivo. The rBMC treatment modalities of some embodiments described herein may be combined with one or more of immune checkpoint inhibitors, recombinantly expressed tumor specific antigens and patient-specific tumor antigens, CAR-T and CAR-N therapies, radiation therapy, and chemotherapeutic drugs.

### 1. rBMC Production

rBMCs are achromosomal, membrane-encapsulated biological nanoparticles (approximately 250-500 nm in diameter depending on the strain type and growth conditions used) that are formed by bacteria following a disruption in the normal cell division apparatus. In essence, rBMCs are small, metabolically active replicas of normal bacterial cells with the exception that they contain no chromosomal DNA and as such, are non-dividing and non-viable. Although rBMCs do not contain chromosomal DNA, plasmid DNA, RNA, native and/or recombinantly expressed proteins, and other metabolites have all been shown to segregate into rBMCs.

Disruptions in the coordination between chromosome replication and cell division lead to rBMC formation from the polar region of most rod-shaped prokaryotes. Disruption of the coordination between chromosome replication and cell division can be facilitated through the over-expression of some of the genes involved in septum formation and binary fission. Alternatively, rBMCs can be produced in strains that harbor mutations in genes involved in septum formation and binary fission. Impaired chromosome segregation mechanisms can also lead to rBMC formation as has been shown in many different prokaryotes.

Similarly, rBMC production can be achieved by the over-expression or mutation of genes involved in the segregation of nascent chromosomes into daughter cells. For example, mutations in the parC or mukB loci of E. coli have been demonstrated to produce rBMCs. Both affect separate requisite steps in the chromosome segregation process in Enterobacteriaceae. It can be assumed that like the cell division genes described above, manipulation of wild type levels of any given gene involved in the chromosome segregation process that result in rBMC production will have similar effects in other family members.

Because the cell division and chromosome replication processes are so critical to survival, there exists a high level of genetic and functional conservation amongst prokaryotic family members with respect to genes responsible for these processes. As a result, the over-expression or mutation of a cell division gene capable of driving rBMC production in one family member can be used to produce rBMCs in another. For example, it has been shown that the over-expression of the E. coli ftsZ gene in other Enterobacteriaceae family members such as Salmonella spp. and Shigella spp as well as other class members such as Pseudomonas spp. will result in similar levels of rBMC production.

The same can be demonstrated in the mutation-based rBMC producing strains of the family Enterobacteriaceae. For example, deletion of the min locus in any of Enterobacteriaceae family members results in rBMC production. Cell division genes from the Enterobacteriaceae in which mutation can lead to rBMC formation include but are not limited to the min genes (MinCDE). While rBMC production from the min mutant strains is possible, these strains have limited commercial value in terms of being production strains. The reason for this is that strains with deletions or mutations within the min genes make rBMCs at constitutively low levels. This presents two problems in terms of commercialization and economies of scale. The first is that rBMC yields from these strains are low, which increases production cost. The second is that rBMC yields are highly variable with mutant strains and lot-to-lot variability has an enormous impact on production cost, manufacturing quality control and regulatory compliance. Using cell division mutant strains to produce rBMCs that encapsulate biologically active molecules such as proteins, RNA, DNA, and other catabolites for diagnostic or therapeutic delivery is problematic. The onset of rBMC production in the mutant strains cannot be controlled and occurs at a low level so that the end result is that some rBMCs will contain no biologically active molecules while others will contain widely variable amounts of biologically active molecules. These shortcomings when taken together or separately greatly restrict the utility of these mutant strains for commercial purposes.

rBMC-producing strains that overexpress cell division genes (“overexpressers”) are preferred over mutation-based strains because the rBMC-production phenotype is controllable if the cell division genes to be overexpressed are placed under the control of an inducible or other conditionally active eubacterial promoter system. rBMC production from strains overexpressing the cell division gene ftsZ were discovered by researchers who were identifying essential cell division genes in E. coli using plasmid-based complementation studies. In these studies, the ftsZ gene was present in over 10 copies per cell. The presence of multiple gene copies of ftsZ was demonstrated to produce rBMCs and extremely long filamented cells. Ultimately, this transition into the irreversible filamentous phenotype negatively impacts rBMC yields from strains overexpressing ftsZ from multi-copy plasmids, although the number of rBMCs produced is still higher than that of any mutant strain. It has since been demonstrated that by reducing the number of ftsZ gene copies to a single, chromosomal duplication, the number of rBMCs produced increases over those strains where ftsZ is located on multi-copy plasmids and that the filamentous phenotype is less profound. Thus, some embodiments described herein include compositions of rBMC-producing strains that inducibly overexpress the ftsZ gene from a duplicate, chromosomally integrated copy of ftsZ. The duplicate ftsZ gene used can be derived directly from the species of bacteria in which the rBMC-production phenotype is being engineered and can also be derived from the ftsZ gene sequence from other species of bacteria. By way of non-limiting example, overexpression of the ftsZ gene of Escherichia coli can be used to generate rBMCs from Escherichia coli and Salmonella typhimurium. Resulting strains include the wild type ftsZ gene and a separate, duplicative, and inducible copy of the ftsZ gene on the chromosome and the inducible genetic suicide mechanism(s) described in U.S. patent publication No. 2010/0112670, which is incorporated herein by its entirety. By way of non-limiting example, division genes that can be over-expressed to produce rBMCs in the family Enterobacteriaceae include but are not limited to ftsZ, minE, sulA, ccdB, and sfiC. In some embodiments, the compositions have a duplicate copy(s) of a cell division gene(s) under the control of an inducible promoter that is stably integrated into the chromosome of a given eubacterial strain. It is easily recognized by one skilled in the art that this same strategy could be imparted if the inducible cell division gene cassette were present on a plasmid, cosmid, bacterial artificial chromosome (BAC), recombinant bacteriophage or other episomal DNA molecule present in the cell.

This inducible phenotype approach to rBMC production has several distinct advantages over mutant systems. The first is that because there are no constitutive genetic mutations in these strains, there exists no selective pressure during normal growth and the cells of the culture maintain a very stable and normal physiology until the rBMC phenotype is induced. The result is that inducible rBMC producing strains are healthier and more stable, which ultimately results in higher yields of rBMCs. Another distinct advantage of using the inducible phenotype approach to rBMC production is in cases where rBMCs are to be used to deliver biologically active molecules such as proteins, therapeutic RNAs, plasmid DNAs, and other bioactive catabolites that can be made by the rBMC-producing parent cells such that the rBMCs that are produced encapsulate those biologically active molecules. In some embodiments, the methods including inducing the formation of the biologically active molecule(s) within the parental cells prior to inducing the rBMC phenotype, so that all the rBMCs produced will contain the desired amount of the biologically active molecule(s). Alternatively, the rBMCs themselves can produce the bioactive molecule after being separated from the parental cells. This includes but is not limited to forming the bioactive molecule from an episomal nucleic acid or RNA encoding for the bioactive molecule located within the rBMC or by preexisting protein constituents of rBMCs after being separated from the parental cells. Any of these expression strategies can be employed to express and display binding moieties on the surfaces of rBMCs. These advantages, when used in combination, result in a higher quality and quantity of rBMCs. In addition, these rBMCs can further include small molecule drugs that can be loaded into rBMCs as described in more detail below.

### 2. rBMC Purification, Formulation, and Sterilization

Because rBMCs are derived from some bacteria that are pathogenic or opportunistically pathogenic, it is of the utmost importance that any contaminating parental cells be functionally eliminated from a given population before administration. Conventionally, live parental cells have been eliminated through either physical means or biological means or both.

Physical means include the use of centrifugation-based separation procedures, filtration methodologies, chromatography methodologies, or any combination thereof.

Biological elimination is achieved by but not limited to the preferential lysis of parental cells, the use of auxotrophic parental strains, treatment with antibiotics, treatment with UV radiation, diaminopimelic acid (DAP) deprivation, selective adsorption of parental cells, treatment with other DNA damaging agents, and induction of a suicide gene.

Preferential lysis of parental cells is typically mediated by inducing the lytic cycle of a lysogenic prophage. In the case of rBMC producing strains, it is most useful to use a prophage that is lysis competent but defective at re-infection, such that rBMCs are not subsequently infected and lysed during activation of the lytic phenotype. Alternatively, and by way of non-limiting example, individual genes such as those classified as members of the holin gene family, can be expressed to achieve similar levels of lysis without the concerns over re-infection inherent to the use of lysogenic prophages. Both approaches are limited by the fact that the lysis event, regardless of the method used to achieve it, expels unacceptable amounts of free endotoxin into the media. Removal of such large amounts of free endotoxin is time consuming, suffers from lot to lot variability, and is ultimately cost prohibitive.

The use of auxotrophic strains raises concerns over reversion and as such can only be used in cases where rBMCs are to be produced from commensal or non-pathogenic strains of bacteria. Thus, their application is limited with respect to being used as a method for elimination of live non-pathogenic parental cells used in rBMC production.

Treatment with UV irradiation can be useful in the elimination of live parental cells on a rBMC production run except for the fact that UV irradiation is considered non-ionizing, is random with respect to its effects on nucleic acids, making results highly variable from lot to lot. In addition, this method is not preferred when using rBMCs to deliver therapeutic or prophylactic nucleic acids as UV irradiation randomly damages all nucleic acids. For instance, plasmid DNA would also be highly susceptible to DNA damage by UV irradiation and may be rendered ineffective although still effectively delivered by rBMCs.

Diaminopimelic acid (DAP) deprivation can be useful in the elimination of live parental cells with the exception that this approach is limited by the number of species it can be used for. In other words, not all parent cell species capable of producing rBMCs require DAP for survival. DAP mutants in E. coli rBMC-producing strains are of great advantage and in some cases preferred over the wild type. The advantage of using DAP is that this compound (diaminopimelic acid, an E. coli cell wall constituent) is critical for the growth of E. coli and is not present in or produced by animals. Thus, should a “viable” E. coli rBMC-producing parental cell be administered along with targeted rBMCs, the parental cell will be unable to grow and will thereby be inert to the animal and with respect to rBMC activity. A similar approach can be used with Salmonella spp. based rBMC-producing parental strains except in that case the aro genes, such as aroB, are removed.

Selective adsorption methodologies have yet to be explored with respect to purifying rBMCs from viable parental cells. Selective adsorption is defined as any process by which parental cells or rBMCs are preferentially adsorbed to a substrate by their affinity for the substrate. By way of non-limiting example, high affinity protein-protein interactions could be exploited for this use. By way of non-limiting example, the novel rBMC outer membrane protein Lpp-OmpA::Protein A has a high affinity for the Fc region of most antibodies. The gene encoding for Lpp-OmpA::Protein A is under the control an inducible promoter could easily be introduced on to the chromosome of an immunomodulatory rBMC producing strain. Immunomodulatory rBMCs could be produced from this strain prior to the activation of expression of the invasin gene such that the rBMCs produced do not express or display Lpp-OmpA::Protein A on their cell surface. Once the desired quantity of immunomodulatory rBMCs is produced from the strain, the viable cells within the culture could be given the signal to produce the Lpp-OmpA::Protein A protein such that Lpp-OmpA::Protein A is only expressed and displayed upon viable cells. Once Lpp-OmpA::Protein A is preferentially expressed on the surface of viable parental cells, they can be easily adsorbed to a substrate coated with antibodies or other Fc-region containing proteins. Once absorbed, rBMCs can be selectively purified away from viable parental cells by a number of different means dependent upon the substrate type used. Substrates include but are not limited to solid-phase chromatographic columns used in gravity filtration applications, magnetic beads, ion exchange columns, or HPLC columns.

In some embodiments, rBMCs are substantially separated from the rBMC-producing parent cells in a composition including rBMCs. For example, after separation, the composition including the rBMCs is more than about 99.9%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 65%, 60%, 55%, 50%, 45%, 40%, 35% or 30% free of rBMC-producing parent cells. In some embodiments, the composition contains less than about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% rBMC-producing parent cells.

In some embodiments, the final composition contains few enough contaminating parental cells, viable or otherwise, so as not to be too toxic or interfere with the activity of targeted rBMCs when administered in vivo for therapeutic purposes.

In some embodiments, rBMC-based biopharmaceuticals formulated for human use, especially parenteral use, conform to sterility under U.S. Pharmacopeia <71>. Embodiments described herein include methods of sterilizing rBMC preparations intended for use in humans by exposure to sterilizing doses of gamma irradiation. Such methods are described in PCT/US2016/045400 and incorporated herein.

### 3. Targeting rBMCs to Specific Cells, Tissues, and Organs

Integrin-targeted rBMCs and integrin-targeted oncolytic rBMCs are used as targeted delivery vehicles to target specific cell types that have elevated expression and/or activity of betal-integrins and are involved in disease in vivo. The targeted integrin-targeted rBMCs disclosed herein are targeted to eukaryotic cancer cell-specific surface antigens that include but are not limited to integrin α2β1, integrin α3β1, integrin α4β1, integrin α5β1, integrin α6β1, integrin αᵥβ1, integrin β4, and integrin β1. As described in more detail below, expression and/or activity levels of these betal integrin family members are found in many solid tumor types as well as in the tumor vasculature as compared to low level, unactivated, and/or ligand occupied betal integrins in normal tissues and vasculature.

Antibody-based targeting of rBMCs and oncolytic rBMCs to tumor or other cells is mediated by the surface display of antibodies or common antibody derivatives, such as single chain antibodies. Several methodologies for functionalizing the surface of rBMCs with targeting antibodies have been described. In one approach, antibodies and antibody derivatives are physically attached to the surface of rBMCs to make them targeting competent. Physical attachment methods include chemical cross-linking, the use of bi-specific antibodies and antibody derivatives, and the attachment of Fc-containing antibodies and antibody derivatives to rBMCs containing the Fc-binding portion of Protein A or Protein G on their surfaces. In some embodiments, the binding portion(s) of Protein A or Protein G is a fusion with the Lpp-OmpA display system which is described in U.S. Pat. Nos. 10,005,820 and 10,919,942, each hereby incorporated by reference in its entirety. The antibody, Fc-containing antibody derivative, and/or Fc-containing fusion/conjugate targeting molecule on the surface of minicells can preferentially recognize but is not limited to recognizing cell-specific surface antigens including β2β1 integrin β3β1 integrin, β4β1 integrin, β5β1 integrin, αᵥβ3 integrin, αᵥβ1 integrin, β4 integrin, β1 integrin, 5T4, CAIX, CD4, CD8, CD11b, CD11c, CD13, CD19, CD20, CD22, CD25, CD30, CD31, CD33, CD34, CD40, CD44v6, CD45, CD51, CD52, CD54, CD56, CD64, CD70, CD74, CD79, CD105, CD117, CD123, CD133, CD138, CD144, CD146, CD152, CD174, CD205, CD227, CD326, CD340, Cripto, ED-B, GD2, TMEFF2, VEGFR1, VEGFR2, FGFR, PDGFR, ANGPT1, TIE1, TIE2, NRP1, TEK (CD202B), TGFβR, Death Receptor 5 (Trail-R2), DLL4, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, EPHB6, FAP, GPNMB, ICAMs, VCAMs, PSMA, HER-2/neu, IL-13R alpha 2, MUC-1, MUC16, EGFR1 (HER-1), EGFR2 (HER-2/neu), EGFR3 (HER-3), IGF-1R, IGF-2R, c-Met (HGFR), Mesothelin, TROP-2, Claudin 18.2, Claudin 6, PDGFR, EDGR, TAG-72, transferrin receptor, EpCAM, CTLA-4, PD-L1, TIGIT, OX40, PSMA, tenascin C, alpha-fetoprotein, vimentin, C242 antigen, TRAIL-R1, TRAIL-R2, CA-125, GPNMB, CA-IX, GD3 ganglioside, RANKL, BAFF, IL-6R, TAG-72, HAMA, and CD166. In some embodiments, the targeting moiety is selected, in part, because the binding of the minicell-surface displayed antibody targeting moiety, Fc-containing antibody derivatives, and/or Fc-containing fusion/conjugate targeting molecules specific for the antigen induce internalization of the targeted minicell, facilitating intracellular payload delivery. Previously described target-specific antibodies that are used as the targeting component, in some embodiments, include but are not limited to mAb 3F8, mAb CSL362, mAb CSL360, mAb J591, Abagovomab, Abciximab, Adalimumab, Afelimomab, Afutuzumab, Alacizumab, ALD518, Alemtuzumab, Altumomab, Anatumomab, Anrukinzumab, Apolizumab, Arcitumomab, Aselizumab, Atlizumab, Atorolimumab, Bapineuzmab, Basiliximab, Bavituximab, Bectumomab, Belimumab, Benralizumab, Bertilimumab, Besilesomab, Bevacizumab, Biciromab, Bivatuzumab, Blinatumomab, Brentuximab, Briakinumab, Canakinumab, Cantuzumab, Capromab, Catumaxomab, CC49, Cedelizumab, Certolizumab, Cetuximab, mAb528, Citatuzumab, Cixutumumab, Clenoliximab, Clivatuzumab, Conatumumab, CR6261, Dacetuzumab, Daclizumab, Daratumumab, Denosumab, Detumomab, Dorlimomab, Dorlixizumab, Ecromeximab, Eculizumab, Edobacomab, Edrecolomab, Efalizumab, Efungumab, Elotuzumab, Elsilimomab, Enlimomab, Epitumomab, Epratuzumab, Erlizumab, Ertumaxomab, Etaracizumab, Exbivirumab, Fanolesomab, Faralimomab, Farletuzumab, Felvizumab, Fezakinumab, Figitumumab, Fontolizumab, Foravirumab, Fresolimumab, Galiximab, Gantenerumab, Gavilimomab, Gemtuzumab, Girentuximab, Glembatumumab, Golimumab, Gomiliximab, Ibalizumab, Irbitumomab, Igovomab, Imciromab, Infliximab, Intetumumab, Inolimomab, Inotuzumab, Ipilimumab, Iratumumab, J591, Keliximab, Labetuzumab, Lebrikizumab, Lemalesomab, Lerdelimumab, Lexatumumab, Libivirumab, Lintuzumab, Lorvotuzumab, Lucatumumab, Lumiliximab, Mapatumumab, Maslimomab, Matuzumab, Mepolizomab, Metelimumab, Milatuzumab, Minretumomab, Mitumomab, Morolimumab, Motavizumab, Muromonab, Nacolomab, Naptumomab, Natalizumab, Nebacumab, Necitutumab, Nerelimomab, Nimotuzumab, Nofetumomab, Ocrelizumab, Odulimomab, Ofatumumab, Olaratumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO140, Rafivirumab, Ramucirumab, Ranibizumab, Raxibacumab, Regavirumab, Resilizumab, Rilotumumab, Rituximab, Robatumumab, Rontalizumab, Rovelizumab, Ruplizumab, Satumomab, Sevirumab, Sibrotuzumab, Sifalimumab, Siltuximab, Siplizumab, Solanezumab, Sonepcizumab, Sontuzumab, Stamulumab, Sulesomab, Tacatuzumab, Tadocizumab, Talizumab, Tanezumab, Taplitumomab, Tefibazumab, Telimomab, Tenatumomab, Teplizumab, TGN1412, Ticilimumab, Tigatuzumab, TNX-650, Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, Zolimomab, and any combination of the preceding.

As an alternative to the physical attachment of antibodies to rBMCs, a host of different recombinant membrane anchored fusion proteins capable of displaying functional single chain antibody fragments on the rBMCs surface may be employed. Methods for making rBMCs targeting competent by addition of antibodies to their surfaces are incorporated by reference to U.S. Pat. No. 7,183,10 and U.S. Ser. No. 13/397,313.

### 4. Loading Payloads into rBMCs

Eubacterial rBMCs are capable of encapsulating and delivering several classes of biologically active compounds that have therapeutic, prophylactic, or diagnostic benefit to an animal. Types of the biologically active compounds (payloads) that can be delivered by rBMCs include but are not limited to small molecules (including small molecule drugs), nucleic acids, polypeptides, radioisotope, lipids, lipopolysaccharides, and any combination thereof.

Proteins include polypeptides and are encoded by DNA. Proteins can be biologically functional, such as enzymes, toxins, or signaling proteins. Proteins can be structural, such as is the case for actin and the like. Proteins can bind tightly to other proteins, such as with antibodies and antibody mimetics, and be used to disrupt functions requiring protein/protein interactions. Proteins can provide localization signals by being fluorescent or bioluminescent. Proteins can serve as immunogens or serve other therapeutic purposes (such as supplying or restoring enzyme in a target cell, tissue, organ, or animal). Proteins can aid in the post-endocytosis intracellular transfer of other payload types. For example, cytolysin proteins such as listeriolysin O (LLO) from Listeria monocytogenes or perfringolysin O (PFO) from Clostridium perfringres can be employed to facilitate the transfer of the rBMC payload(s) from the endocytic compartment(s) of a target cell to the cytosol of a target cell. Proteins can also be pro-drug converting enzymes such as thymidine kinase or cytosine deaminase.

Recombinantly expressed/produced therapeutic polypeptides to be delivered by targeted rBMCs include but are not limited to protein toxins, cholesterol-dependent cytolysins, functional enzymes, antibody mimetics, protein/protein interaction disrupters, activated caspases, pro-caspases, cytokines, chemokines, cell-penetrating peptides, and any combination of the proceeding. Recombinant expression of a therapeutic polypeptide(s) can be the result of expression from any of the various episomal recombinant prokaryotic expression vectors known in the art including but not limited to plasmids, cosmids, phagemids, and bacterial artificial chromosomes (BACs), and any combination of the preceding. In similar fashion, recombinant expression can be achieved by a chromosomally located prokaryotic expression cassette present in one or more copies of the rBMC-producing parent cell chromosome. The delivery of protein toxins using the targeted rBMCs disclosed herein is a particularly attractive approach in applications where selective elimination of cells in vivo is desirable. Protein toxins include but are not limited to gelonin, diphtheria toxin fragment A, diphtheria toxin fragment A/B, tetanus toxin, E. coli heat labile toxin (LTI and/or LTII), cholera toxin, C. perfringes iota toxin, Pseudomonas exotoxin A, shiga toxin, anthrax toxin, MTX (B. sphaericus mosquilicidal toxin), streptolysin, barley toxin, mellitin, anthrax toxins LF and EF, adenylate cyclase toxin, botulinolysin B, botulinolysin E3, botulinolysin C, botulinum toxin A, cholera toxin, clostridium toxins A, B, and alpha, ricin, shiga A toxin, shiga-like A toxin, cholera A toxin, pertussis S1 toxin, and E. coli heat labile toxin (LTB).

Cytolysins are a related family of cholesterol dependent pore forming proteins that include but are not limited to pH stable variants of listeriolysin O (pH-independent; amino acid substitution L461T), thermostable variants of listeriolysin O (amino acid substitutions E247M, D320K), pH and thermostable variants of listeriolysin O (amino acid substitutions E247M, D320K, and L461T), streptolysin O, streptolysin O c, streptolysin O e, sphaericolysin, anthrolysin O, cereolysin, thuringiensilysin O, weihenstephanensilysin, alveolysin, brevilysin, butyriculysin, tetanolysin O, novyilysin, lectinolysin, pneumolysin, mitilysin, pseudopneumolysin, suilysin, intermedilysin, ivanolysin, seeligeriolysin O, vaginolysin, and pyolysin. Cytolysins facilitate endosomal escape and also serve to facilitate oncolysis, although the latter is not a requirement of cytolysins. The preferred method for incorporating cytolysin proteins into rBMCs is via recombinant expression of the cytolysin (for example, to generate a pre-formed cytolysin that is encapsulated in the rBMC).

Recombinant proteins may be localized to different sub-cellular compartments of the rBMC at the discretion of the artisan. When targeted rBMCs disclosed herein are derived from a Gram-negative parental rBMC-producing strain, recombinantly expressed therapeutic polypeptides produced therefrom can be localized to the cytosol, the inner leaflet of the inner membrane, the outer leaflet of the inner membrane, the periplasm, the inner leaflet of the outer membrane, the outer membrane of rBMCs, and any combination of the proceeding. When targeted rBMCs disclosed herein are derived from a Gram-positive parental rBMC-producing strain, recombinantly expressed therapeutic polypeptides produced therefrom can be localized to the cytosol, the cell wall, the inner leaflet of the membrane, the membrane of rBMCs, and any combination of the proceeding.

Small molecules and nucleic acids, both include but are not limited to cytosolic nucleic acid receptors, such as STING and/or RIG-I, can be loaded into purified rBMCs by coincubation with said small molecule or nucleic acid in a suitable medium over a period of time. The preferred temperature range is from 50° C. to 1° C. and the preferred incubation time ranges from 30 minutes to 3 days, although the latter may be extended to suit the needs of the molecule to be loaded at the discretion of the practicing skilled artisan. The concentration of the small molecule or nucleic acid is at the discretion of the artisan and can be modulated to suit the intended and desired therapeutic effect of the composition. Generally speaking, the preferred small molecule or nucleic acid concentration ranges from 1 femtograms/mL to 1,000 milligrams/mL depending on the solubility and other factors known to the artisan. The loading solvent is also quite modular with respect to polarity, but generally speaking should be relatively physiologically isotonic and with a buffered pH range from 4.0 to 9.0. Exhaustive methods on how to load rBMCs with various small molecules and nucleic acids are disclosed in the public domain for the skilled artisan to rely upon. More than one small molecule or nucleic acid species can be loaded simultaneously by admixing prior to incubation or serially at the discretion of the artisan. In some embodiments, purified rBMCs comprising a pre-formed endosomal escape protein are co-incubated with a STING and/or RIG-I agonist for at least 2 hours at 4° C. In the examples provided herein, 10¹⁰ rBMCs were loaded with 30 μg/mL of ADU-S100 for 18 hours at 4° C.

### 5. Pharmaceutical Compositions

The present application also relates to compositions, including but not limited to pharmaceutical compositions. The term “composition” used herein refers to a mixture including at least one carrier, for example, a physiologically acceptable carrier, and one or more rBMC compositions. The term “carrier” used herein refers to a chemical compound that does not inhibit or prevent the incorporation of the biologically active peptide(s) into cells or tissues. A carrier typically is an inert substance that allows an active ingredient to be formulated or compounded into a suitable dosage form (e.g., a pill, a capsule, a gel, a film, a tablet, a microparticle (e.g., a microsphere), a solution; an ointment; a paste, an aerosol, a droplet, a colloid or an emulsion etc.). A “physiologically acceptable carrier” is a carrier suitable for use under physiological conditions that does not abrogate (reduce, inhibit, or prevent) the biological activity and properties of the compound. For example, dimethyl sulfoxide (DMSO) is a carrier as it facilitates the uptake of many organic compounds into the cells or tissues of an organism. In some embodiments, the carrier is a physiologically acceptable carrier, including, for example, a pharmaceutically or veterinarily acceptable carrier, in which the rBMC composition is disposed.

A “pharmaceutical composition” refers to a composition wherein the carrier is a pharmaceutically acceptable carrier, while a “veterinary composition” is one wherein the carrier is a veterinarily acceptable carrier. The term “pharmaceutically acceptable carrier” or “veterinarily acceptable carrier” used herein includes any medium or material that is not biologically or otherwise undesirable, e.g., the carrier may be administered to an organism along with a rBMC composition without causing any undesirable biological effects or interacting in a deleterious manner with the complex or any of its components or the organism. Examples of pharmaceutically acceptable reagents are provided in The United States Pharmacopeia, The National Formulary, United States Pharmacopeial Convention, Inc., Rockville, Md. 1990, hereby incorporated by reference herein into the present application. The terms “therapeutically effective amount” and “pharmaceutically effective amount” refer to an amount sufficient to induce or effectuate a measurable response in the target cell, tissue, or body of an organism. What constitutes a therapeutically effective amount will depend on a variety of factors, which the knowledgeable practitioner will consider in arriving at the desired dosage regimen.

The compositions can also include other chemical components, such as diluents and excipients. A “diluent” is a chemical compound diluted in a solvent, such as an aqueous solvent, that facilitates dissolution of the composition in the solvent, and it may also serve to stabilize the biologically active form of the composition or one or more of its components. Salts dissolved in buffered solutions are utilized as diluents in the art. In some embodiments, diluents are buffered solutions containing one or more different salts. An unlimiting example of a buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a given compound or pharmaceutical composition.

An “excipient” is any inert substance that can be added to a composition to confer a suitable property, for example, a suitable consistency or to produce a drug formulation. Suitable excipients and carriers include fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, methyl cellulose, hydroxypropylmethyl-cellulose, polyacrylate, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See e.g., WO 98/52547 (which describes microsphere formulations for targeting compounds to the stomach, the formulations including an inner core (optionally including a gelled hydrocolloid) containing one or more active ingredients, a membrane having a water insoluble polymer (e.g., ethylcellulose) to control the release rate of the active ingredient(s), and an outer layer including a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

The compositions can be formulated in any suitable manner. rBMC compositions may be uniformly (homogeneously) or non-uniformly (heterogeneously) dispersed in the carrier. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other exemplary dry formulations include those wherein a composition disclosed herein is compressed into tablet or pill form suitable for oral administration or compounded into a sustained release formulation. When the composition is intended for oral administration to be delivered to epithelium in the intestines, an exemplary formulation can be encapsulated with an enteric coating to protect the formulation and prevent premature release of the rBMC compositions included therein. As those in the art will appreciate, the compositions disclosed herein can be placed into any suitable dosage form. Pills and tablets represent some of such dosage forms. The compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some embodiments provide compositions that include a bioadhesive, such as a mucoadhesive, coating. A “bioadhesive coating” is a coating that allows a substance (e.g., a rBMC composition) to adhere to a biological surface or substance better than occurs absent the coating. A “mucoadhesive coating” is an exemplary bioadhesive coating that allows a substance, for example, a composition to adhere better to mucosa occurs absent the coating. For example, rBMCs can be coated with a mucoadhesive. The coated particles can then be assembled into a dosage form suitable for delivery to an organism. In some embodiments, and depending upon the location where the cell surface transport moiety to be targeted is expressed, the dosage form is then coated with another coating to protect the formulation until it reaches the desired location, where the mucoadhesive enables the formulation to be retained while the composition interacts with the target cell surface transport moiety.

Compositions disclosed herein can be administered to any organism, such as an animal, including, for example, a mammal, bird, fish, insect, or arachnid. Exemplary mammals include bovine, canine, equine, feline, ovine, and porcine animals, and non-human primates. In some embodiments, the mammal is a human. Multiple techniques of administering or delivering a compound exist in the art including, but not limited to, oral, intraocular, intracranial, rectal (e.g. an enema or suppository) aerosol (e.g., for nasal or pulmonary delivery), topical administration (including intravesical administration to the urinary bladder), and parenteral, including but not limited to subcutaneous, intradermal, intratumoral, and intravenous routs of administration. In some embodiments, sufficient quantities of the biologically active peptide are delivered to achieve the intended effect. The particular amount of composition to be delivered will depend on many factors, including the effect to be achieved, the type of organism to which the composition is delivered, delivery route, dosage regimen, and the age, health, and sex of the organism. As such, the particular dosage of a composition incorporated into a given formulation is left to the ordinarily skilled artisan's discretion.

Those skilled in the art will appreciate that when the compositions disclosed herein are administered as agents to achieve a particular desired biological result, which may include a therapeutic, diagnostic, or protective effect(s) (including vaccination), it may be possible to combine the rBMC composition with a suitable pharmaceutical carrier. The choice of pharmaceutical carrier and the preparation of the rBMCs as a therapeutic or protective agent will depend on the intended use and mode of administration. Suitable formulations and methods of administration of therapeutic agents include those for oral, pulmonary, nasal, buccal, ocular, dermal, rectal, intravenous, or vaginal delivery.

Depending on the mode of delivery employed, the context-dependent functional entity can be delivered in a variety of pharmaceutically acceptable forms. For example, the context-dependent functional entity can be delivered in the form of a solid, solution, emulsion, dispersion, and the like, incorporated into a pill, capsule, tablet, suppository, aerosol, droplet, or spray. Pills, tablets, suppositories, aerosols, powders, droplets, and sprays may have complex, multilayer structures and have a large range of sizes. Aerosols, powders, droplets, and sprays may range from small (1 micron) to large (200 micron) in size.

Pharmaceutical compositions disclosed herein can be used in the form of a solid, a lyophilized powder, a solution, an emulsion, a dispersion, and the like, wherein the resulting composition contains one or more of the compounds disclosed herein, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary, stabilizing, thickening, and coloring agents and perfumes may be used. Examples of a stabilizing dry agent include triulose, for example, at concentrations of 0.1% or greater (See, e.g., U.S. Pat. No. 5,314,695). The active compound is included in the pharmaceutical composition in an amount sufficient to produce the desired effect upon the process or condition of diseases.

In some embodiments, rBMC-based biopharmaceuticals formulated for human use, especially parenteral use, should conform to sterility under U.S. Pharmacopeia <71>. Embodiments described herein include methods of sterilizing rBMC preparations intended for use in humans by exposure to sterilizing doses of gamma irradiation. Such methods are described in PCT/US2016/045400 and incorporated herein.

### 6. Therapeutic Indications

The present application relates to dual STING/RIG-I agonist oncolytic rBMC-mediated in situ immunization, especially CTL responses, against cancer types including but not limited to solid tumors, metastatic tumors, and liquid tumors. Solid and metastatic tumors include those of epithelial, fibroblast, muscle and bone origin and include but are not limited to breast, lung, pancreatic, prostatic, testicular, ovarian, gastric, intestinal, mouth, tongue, pharynx, hepatic, anal, rectal, colonic, esophageal, urinary bladder, gall bladder, skin, uterine, vaginal, penal, and renal cancers. Other solid cancer types that may be treated with the dual STING/RIG-I agonist oncolytic rBMCs disclosed herein include but are not limited to adenocarcinomas, sarcomas, fibrosarcomas, and cancers of the eye, brain, and bone. Liquid tumors that can be treated by the dual STING/RIG-I agonist oncolytic rBMCs disclosed herein include but are not limited to non-Hodgkin's lymphoma, myeloma, Hodgkin's lymphoma, acute lymphocytic leukemia, chronic lymphocytic leukemia, acute myeloid leukemia, chronic myeloid leukemia, and other leukemias.

### 7. rBMC Preparations

Some embodiments relate to creating an optimized strain and preparing immunomodulatory rBMCs from, but not limited to, the bacterial family Enterobacteriaceae.

In some embodiments, the level of rBMC producing viable parental cell contamination is less than 1 in 10⁵ rBMCs. In some embodiments, the level of rBMC producing viable parental cell contamination is less than 1 in 10⁶ rBMCs.

In some embodiments, the level of rBMC producing viable parental cell contamination is less than 1 in 10⁷ rBMCs.

In some embodiments, the level of rBMC producing viable parental cell contamination is less than 1 in 10⁸ rBMCs.

In some embodiments, the level of rBMC producing viable parental cell contamination is less than 1 in 10⁹ rBMCs.

In some embodiments, the level of rBMC producing viable parental cell contamination is less than 1 in 10¹⁰ rBMCs.

In some embodiments, the level of rBMC producing viable parental cell contamination is less than 1 in 10¹¹ rBMCs.

In some embodiments, the level of rBMC producing viable parental cell contamination is less than 1 in 10¹² rBMCs.

In some embodiments, the level of rBMC producing viable parental cell contamination is less than 1 in 10¹³ rBMCs.

In some embodiments, the level of rBMC producing viable parental cell contamination is less than 1 in 10¹⁴ rBMCs.

In some embodiments, the level of rBMC producing viable parental cell contamination is less than 1 in 10¹⁵ rBMCs.

In some embodiments, the level of rBMC producing viable parental cell contamination is less than 1 in 10¹⁶ rBMCs.

Some embodiments are provided in the following enumerated alternatives.

1. A recombinant bacterial minicell (rBMC) for the inhibition or treatment of cancer, comprising: invasin; perfringolysin O (PFO); and a stimulator of interferon genes (STING) agonist or a retinoic acid inducible gene I (RIG-I) agonist.

2. A recombinant bacterial minicell (rBMC) for the inhibition or treatment of cancer, comprising: a surface localized targeting molecule; a cytolysin protein; and an agonist of an intracellular mediator of a Type I IFN response.

3. The rBMC of alternative 2, wherein the surface localized targeting molecule comprises invasin.

4. The rBMC of any one of alternatives 2-3, wherein the cytolysin protein comprises perfringolysin O (PFO).

5. The rBMC of any one of alternatives 2-4, wherein the agonist is a STING agonist, a RIG-I agonist, or both.

6. The rBMC of alternative 5, wherein the STING agonist comprises c-di-GMP, 2′3′cGAMP, 3′3′cGAMP, cAIMP, cAIMP difluoro, cAIMP (PS) 2 Difluor (Rp/Sp), a cyclic di-nucleotide, ADU-S100, MK-1454, BMS-986301, E7766, GSK3745417, SB 11285, or TAK-676, or any combination thereof.

7. The rBMC of any one of alternatives 5-6, wherein the RIG-I agonist comprises an uncapped 5′triphosphate RNA.

8. The rBMC of alternative 7, wherein the 5′triphosphate RNA ranges from about 30 to about 2,000 nucleotides in length.

9. The rBMC of any one of alternatives 7-8, wherein the 5′triphosphate RNA is single stranded or double stranded.

10. The rBMC of any one of alternatives 7-9, wherein the RIG-I agonist comprises polyI:C, SLR14, polyU/UC, RN7SL1, M8, 3p-siBCL2, MK-4621, and BO-112.

11. The rBMC of any one of alternatives 1-10, wherein the rBMC expresses one or more recombinant tumor selective antigens.

12. The rBMC of alternative 11, wherein the one or more recombinant tumor selective antigens is HER-2, K-RAS, H-RAS, N-RAS, MAGE, c-MYC, MUC-1, PSMA, CEA, ETA, CA-125, p53, AFP, Tyrosinase, oncofetal protein, or antigens produced by oncogenic viruses.

13. The rBMC of any one of alternatives 1-12, wherein the rBMC is produced from a naturally invasive strain of bacteria.

14. The rBMC of alternative 13, wherein the naturally invasive strain of bacteria comprises Salmonella spp., Listeria spp., Mycobacterium spp., Shigella spp., Yersinia spp., or Escherichia coli.

15. The rBMC of any one of alternatives 1-14, wherein the rBMC is an oncolytic rBMC.

16. A pharmaceutical composition comprising: a recombinant bacterial minicell as recited in any one of alternatives 1-15; and a pharmaceutically acceptable carrier.

17. The pharmaceutical composition of alternative 16, further comprising an immune checkpoint inhibitor.

18. The pharmaceutical composition of alternative 17, wherein the immune checkpoint inhibitor comprises an inhibitor against PD-1, PD-L1, PD-L2, PD-L3, PD-L4, CTLA-4, LAG-3, IDO, B7-H3, B7-H4, GITR, 4-1BB, OX40, CD27, KIR2DL, CSF1R, CD40L, KIR, or TIM-3.

19. A method of inhibiting or treating cancer, comprising: administering to a subject having cancer a composition comprising the rBMC of any one of alternatives 1-15 or the pharmaceutical composition of any one of alternatives 16-18.

20. A method of inhibiting or treating cancer, comprising: administering to a subject having cancer a pharmaceutical composition comprising a targeted oncolytic recombinant bacterial minicell (rBMC), wherein the bacterial rBMC is configured to stimulate upstream intracellular mediators of a Type I IFN response, wherein the intracellular mediators of the Type I IFN response comprise stimulator of interferon genes (STING) and retinoic acid inducible gene I (RIG-I) nucleic acid sensing pathways.

21. The method of alternative 20, wherein said method inhibits the growth of cancer.

22. The method of any one of alternatives 20-21, wherein the method inhibits or delays the onset of cancer.

23. The method of any one of alternatives 19-22, wherein the cancer is a solid tumor, a metastatic tumor, or a liquid tumor.

24. The method of any one of alternatives 19-23, wherein the cancer is epithelial, fibroblast, muscle, or bone origin.

25. The method of any one of alternatives 19-24, wherein the cancer is adenocarcinoma, sarcoma, fibrosarcoma, eye, brain, bone, breast, lung, pancreatic, prostatic, testicular, ovarian, gastric, intestinal, mouth, tongue, pharynx, hepatic, anal, rectal, colonic, esophageal, urinary bladder, gall bladder, skin, uterine, vaginal, penal, renal cancer, non-Hodgkin's lymphoma, myeloma, Hodgkin's lymphoma, acute lymphocytic leukemia, chronic lymphocytic leukemia, acute myeloid leukemia, or chronic myeloid leukemia.

26. The method of any one of alternatives 19-25, further comprising administering an immune checkpoint inhibitor therapy.

27. The method of alternative 26, wherein the immune checkpoint inhibitor therapy comprises administration of one or more immune checkpoint inhibitors.

28. The method of alternative 27, wherein the one or more immune checkpoint inhibitors comprises an inhibitor against PD-1, PD-L1, PD-L2, PD-L3, PD-L4, CTLA-4, LAG-3, IDO, B7-H3, B7-H4, GITR, TIGIT, 4-1BB, OX40, CD27, KIR2DL, CSF1R, CD40L, KIR, or TIM-3.

29. The method of any one of alternatives 20-28, wherein the rBMC comprises a surface localized targeting molecule.

30. The method of alternatives 29, wherein the surface localized targeting molecule comprises invasin.

31. The method of any one of alternatives 20-30, wherein the rBMC comprises a cytolysin protein.

32. The method of alternative 31, wherein the cytolysin protein is perfringolysin O (PFO).

33. The method of any one of alternatives 20-32, wherein the rBMC comprises a STING agonist, a RIG-I agonist, or both.

34. The method of alternative 33, wherein the STING agonist comprises c-di-GMP, 2′3′cGAMP, 3′3′cGAMP, cAIMP, cAIMP difluoro, cAIMP (PS) 2 Difluor (Rp/Sp), a cyclic di-nucleotide, ADU-S100, MK-1454, BMS-986301, E7766, GSK3745417, SB 11285, or TAK-676, or any combination thereof.

35. The method of any one of alternatives 33-34, wherein the RIG-I agonist comprises an uncapped 5′triphosphate RNA.

36. The method of alternative 35, wherein the 5′triphosphate RNA ranges from about 30 to about 2,000 nucleotides in length.

37. The method of any one of alternatives 35-36, wherein the 5′triphosphate RNA is single stranded or double stranded.

38. The method of any one of alternatives 35-37, wherein the RIG-I agonist comprises polyI: C, SLR14, polyU/UC, RN7SL1, M8, 3p-siBCL2, MK-4621, and BO-112.

39. The method of any one of alternatives 20-38, wherein the rBMC expresses one or more recombinant tumor selective antigens.

40. The method of alternative 39, wherein the one or more recombinant tumor selective antigens is HER-2, K-RAS, H-RAS, N-RAS, MAGE, c-MYC, MUC-1, PSMA, CEA, ETA, CA-125, p53, AFP, Tyrosinase, oncofetal protein, or antigens produced by oncogenic viruses.

41. The method of any one of alternatives 20-40, wherein the rBMC is produced from a naturally invasive strain of bacteria.

42. The method of alternative 41, wherein the naturally invasive strain of bacteria comprises Salmonella spp., Listeria spp., Mycobacterium spp., Shigella spp., Yersinia spp., or Escherichia coli.

In some embodiments of the methods described herein, any of the methods described herein can be used alone, or any of the methods described herein can be used in combination with any other method or methods described herein.

Unless defined otherwise, all technical and scientific terms used herein have the same meaning as commonly understood by one of ordinary skill in the art. Although the present application has been described with reference to embodiments and examples, it should be understood that various modifications can be made without departing from the spirit of the disclosure. All references cited herein are expressly incorporated herein by reference in their entirety.

### EXAMPLES

While the present disclosure has been described in some detail for purposes of clarity and understanding, one skilled in the art will appreciate that various changes in form and detail can be made without departing from the true scope of the embodiments described herein.

### Example 1: STING-Mediated Activation of Type I IFN in Response to VAX014

This example demonstrates that the compositions described herein trigger production of Type I IFN in bladder cancer cells.

As the search for better cancer treatments advances, recent insights highlight antigen presentation, T cell activation and Type I interferon (IFN) as critical steps to develop antitumor immunity, which significantly improves patient outcomes in many cancers. Tumors adapt to escape immune control by, for example, upregulating checkpoint molecules like PD-L1, downregulating antigen presenting molecules like MHC-I, and dysregulating the Type I IFN response. Some therapies activate antitumor immunity but paradoxically are susceptible to Type I IFN, which may explain why clinical responses have thus far been limited. Oncolytic therapies that are not susceptible to Type I IFN are needed. Innate immune mechanisms that produce IFN include the stimulator of IFN genes (STING) and retinoic acid-inducible gene-I (RIG-I) pathways.

An oncolytic bacterial rBMC composition, VAX014, was prepared. VAX014 is a novel oncolytic agent that originates from E. coli rBMCs. In this example, ELISA was used to show that VAX014 triggers production of Type I IFN in MB49 cells (p<0.0001). Using culture supernatants, an antibody that blocks the Type I IFN receptor, and flow cytometry, IFN acts in autocrine/paracrine fashion to upregulate both PD-L1 (p<0.0001) and MHC-I (p<0.0001) in vitro. Western blots revealed that MB49 cells express STING and STING function was confirmed using the murine STING agonist, DMXAA, which stimulated IFN production (p<0.0001), as shown in Appendix I.

To investigate if VAX014 activates the STING pathway, MB49 cells were pre-treated with a selective, irreversible murine STING inhibitor, H-151. A reduction of PD-L1 (p<0.05) and MHC-I expression was observed after treatment with VAX014. These results indicate that VAX014 activates multiple pathways in MB49, including STING, to induce production of Type I IFN. Taken together, these results suggest that VAX014 may engage several mechanisms of immune activation in cancer, including Type I IFNs, leading to better outcomes for patients.

### Example 2: Treatment of Bladder Cancer Cells with Oncolytic Bacterial rBMCs

This example demonstrates the efficacy of treating bladder cancer cells with the compositions described herein.

An oncolytic rBMC composition, VAX014, was prepared. VAX014 is a novel oncolytic agent that originates from E. coli rBMCs. With the use of invasin, it targets cells that express α3β1 or α5β1 integrins, both of which are overexpressed in tumors of late-stage bladder cancer patients. VAX014 is then endocytosed by the cell and degraded to release perfringolysin O, a membrane pore-forming protein toxin, resulting in oncolysis. VAX014 has strong effects in mouse models of bladder cancer, including the orthotopic MB49 model.

Bladder cancer is prevalent in the United States, being the 4th and 8th most common type of cancer in men and women, respectively. These tumors avoid mechanisms of immunity in several ways, including disrupting interferon (IFN) production which is required for antitumor adaptive immunity to develop. Type I IFNs can be produced through the retinoic acid-inducible gene-I (RIG-I) pathway upon recognition of foreign RNA in the cytoplasm. VAX014 has shown strong immune-dependent antitumor effects in the orthotopic MB49 mouse model of bladder cancer. In this example, the role of RIG-I in the response to VAX014 in the MB49 model was investigated. Western blotting was used to confirm RIG-I expression and ELISA on culture supernatants to confirm the presence of Type I IFNs after treatment with VAX014. CRISPR/Cas9 was used to knock out the RIG-I gene in MB49 cells. In this system, a CRISPR/Cas9 plasmid directs the Cas9 enzyme to make double stranded cuts at the site of the RIG-I gene. A second plasmid repairs the breaks by inserting into the genomic DNA, silencing the RIG-I gene. Knockout of RIG-I is indicated by expression of RFP and puromycin resistance. MB49 cells were grown until confluent and transfected with both plasmids. RFP expression observed via microscopy confirmed successful transfection and cells underwent puromycin selection at 7.5 μg/mL. Transfected cells grew to confluency, while mock- and un-transfected controls died, indicating that the transfected cells were puromycin resistant. Once confluent, resistant cells were transferred to 96-well plates for clonal selection by limiting dilution. Wild-type MB49 cells were also plated in puromycin and grown alongside experimental plates. 81 colonies were obtained from the plasmid transfected plates, while zero were obtained from the wild-type cells.

When treated with VAX014, MB49 cells show upregulation of proteins that are integral to T cell responses, like PD-L1 and MHC-I. PD-L1 acts as a checkpoint that keeps cytotoxic T cells under control while MHC-I is a cell surface receptor family that aids in T-cell recognition. Both PD-L1 and MHC-I upregulation can be caused by increased Type I interferon (IFN) production, as shown in Appendix II.

Isolated clones undergo expansion and select clones are re-tested for puromycin resistance and probed for RIG-I absence via Western blot. With the successful knockout of RIG-I in MB49 cells, the response of RIG-I negative MB49 to VAX014 is evaluated in vitro and in vivo, to determine if the absence of RIG-I has substantial effect on Type I IFN production or tumor activity following treatment with VAX014.

### Example 3: Oncolytic Bacterial rBMCs Activate Type I IFN in a PFO Dependent Matter

This example demonstrates that incorporating an endosomal escape protein is critical for Type I IFN production and that the activation is mediated through the dual activation of STING and another TBK-1 dependent Type I IFN pathway.

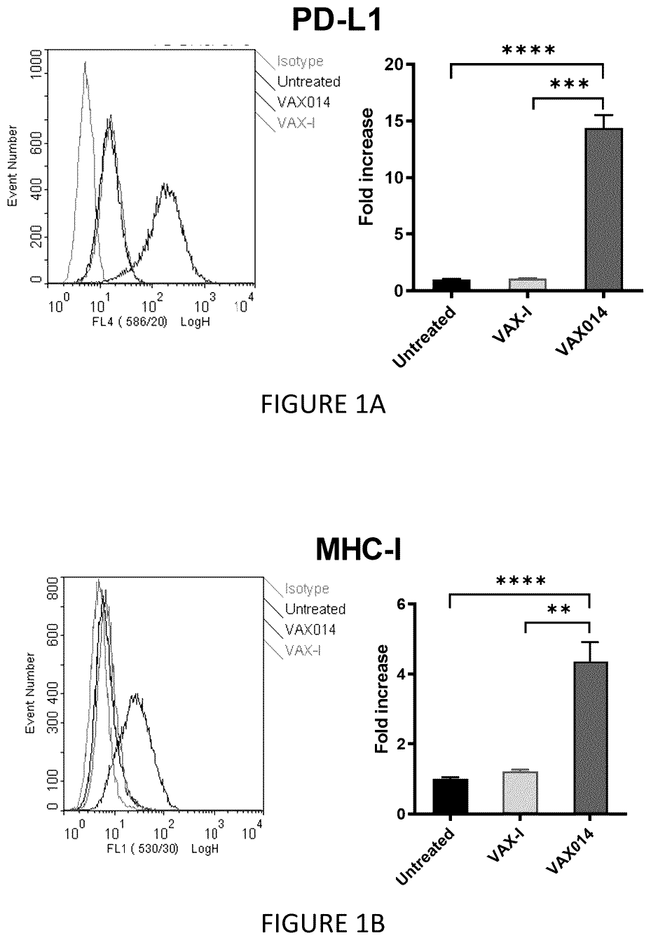

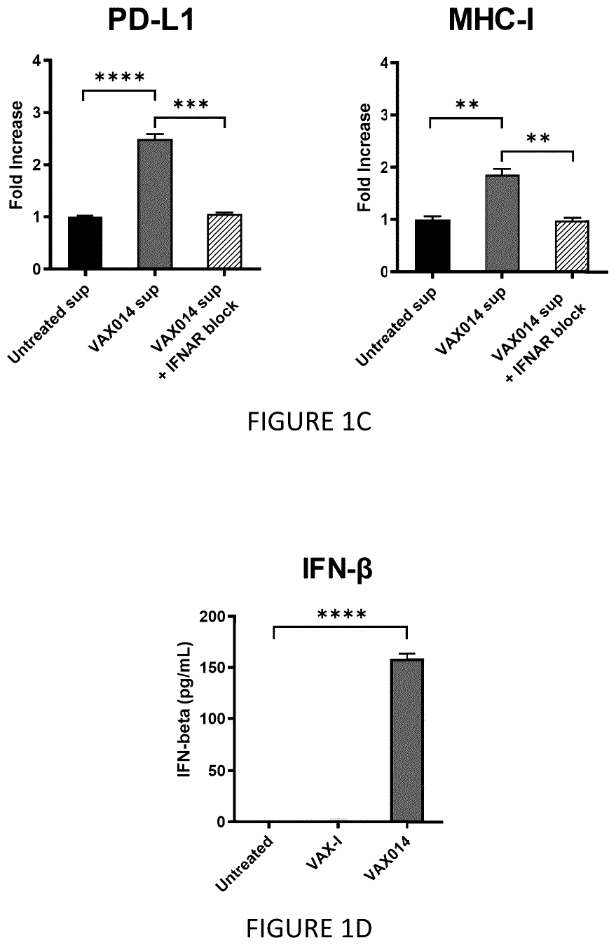

No Type I IFN was observed when the endosomal escape protein was absent. Murine MB49 urothelial carcinoma tumor cell line upregulates programmed death ligand 1 (PD-L1) and major histocompatibility complex I (MHCI) (determined by flow cytometry) in a PFO dependent manner (FIGS. 1A and 1B). VAX-I is a control minicell containing no PFO endosomal escape protein. PD-L1 and MHC-I (FIG. 1C) upregulation is dependent upon Type I IFN as indicated by a loss of upregulation when naïve MB49 cells have been pre-incubated with a receptor function blocking antibody against IFNAR1 (the sole receptor for Type I IFN) before transfer of VAX014 treated supernatants. IFN-β, detected by ELISA, is present in supernatants of VAX014 treated MB49 cells (FIG. 1D), but not those of VAX-I treated cells, indicating a need for PFO-mediated endosomal escape to elicit Type I IFN production.

### Example 4: Oncolytic Bacterial rBMCs Activate STING and RIG-I Pathways

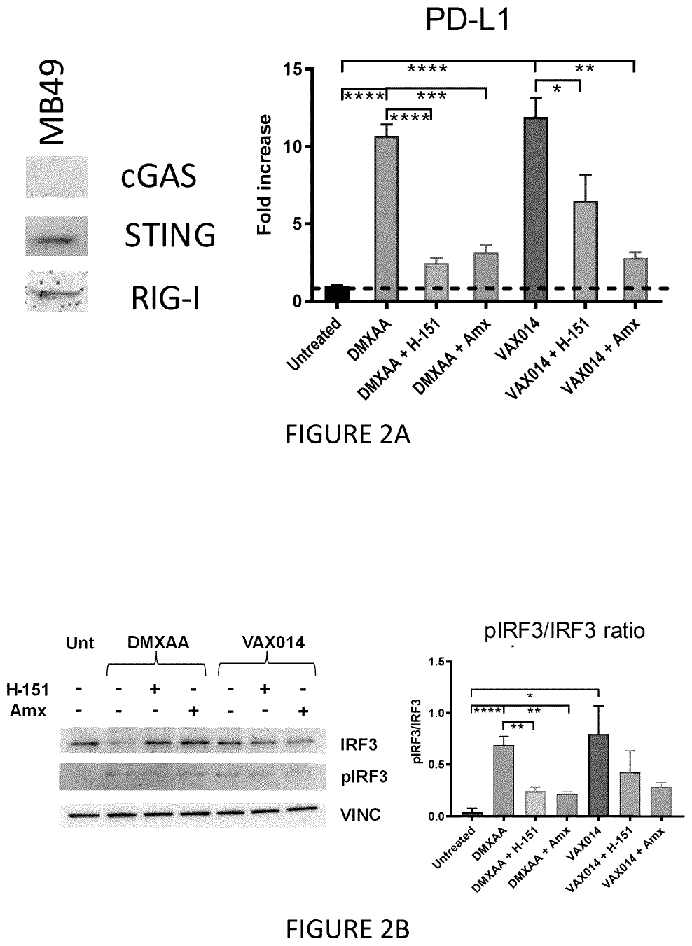

Wild type MB49 cells were found to express STING and RIG-I by Western blot. MB49 cells do not express cGAS, the cytosolic enzyme that recognizes, binds to, and converts cytosolic DNA into 2′-3′cGAMP (a unique non-prokaryotic cyclic dinucleotide) (FIG. 2A). Due to a lack of cGAS, MB49 is the ideal system in which to evaluate the effect of cyclic dinucleotide delivery in the absence of contributing noise from cGAS-mediated 2′-3′cGAMP production. VAX014 activates the STING pathway in WT MB49 as indicated by a drop in PD-L1 expression in the presence of H-151 (an irreversible STING inhibitor). However, H-151 did not eliminate PD-L1 activity completely, suggesting activation of at least one other pathway. Preincubation with Amlexanox (AMX), a competitive TBK-1 inhibitor, led to a drop approaching baseline expression, indicating VAX014 activates more than one TBK-1 mediated Type I IFN signaling pathway. In contrast and as a control, DMXAA (a murine STING agonist with excellent cell permeability) was able to induce PD-L1 expression that could be brought completely to baseline by the addition of either H-151 or TBK-1 (thereby indicating activation of only the STING pathway with DMXAA). This was orthogonally confirmed by evaluating the activated phospho-IRF3 (pIRF3) to total IRF3 ratio in cell lysates obtained under identical conditions (FIG. 2B).

### Example 5: Genetic Ablation of STING Confirms that Oncolytic Bacterial rBMCs Activate the STING Pathways

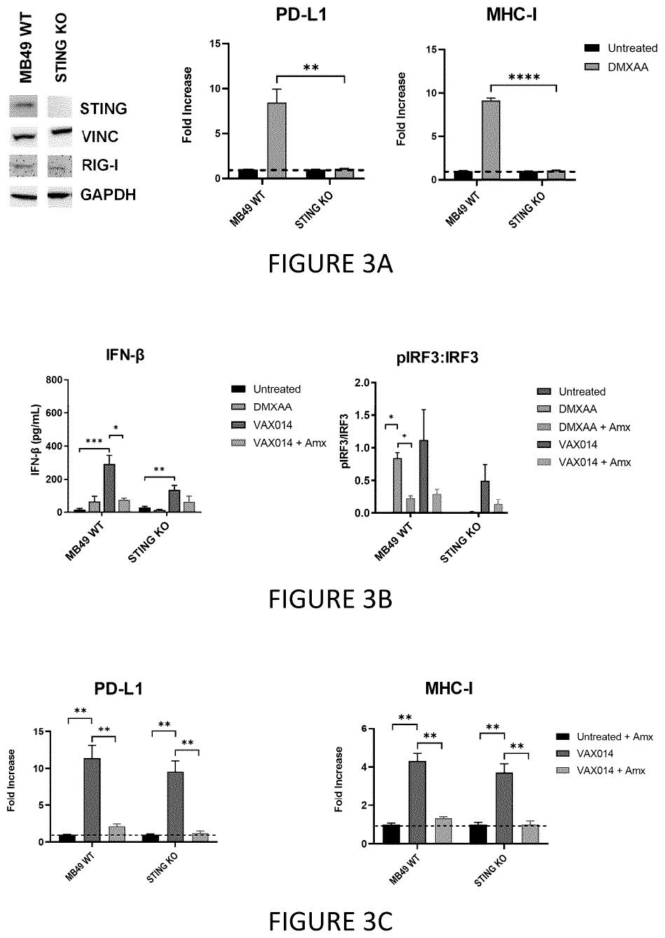

Genetic ablation of STING confirmed the findings in Example 4, while genetic ablation of RIG-I confirmed the additional activation of the RIG-I pathway (Example 6). A biallelic targeted deletion of the murine STING gene was generated using a commercially available CRISPR-Cas9 kit to create a MB49-STING KO cell line. Deletion of STING and the preservation of RIG-I expression was confirmed by Western blot (FIG. 3A). Loss of STING function in the MB49-STING KO line was confirmed by a loss of PD-L1 and MHC-I in response to treatment with DMXAA (FIG. 3A). In contrast, VAX014 still maintained the ability to upregulate IFN-β at a detectable level in the MB49-STING KO cell line (FIG. 3B). Commensurate reductions in the pIRF3/IRF3 ratio (FIG. 3B) and reduced surface expression of PD-L1 and MHC-I were also observed (FIG. 3C). Preincubation with AMX lowered each of IFN-β production, pIRF3/IRF3 ratios, and PD-L1/MHC-I surface expression nearly to baseline conditions of the untreated control, thus confirming the preservation of the alternate non-STING TBK-1 mediated pathway induced by VAX014.

### Example 6: Genetic Ablation of RIG-I Confirms that Oncolytic Bacterial rBMCs Activate the RIG-I Pathway

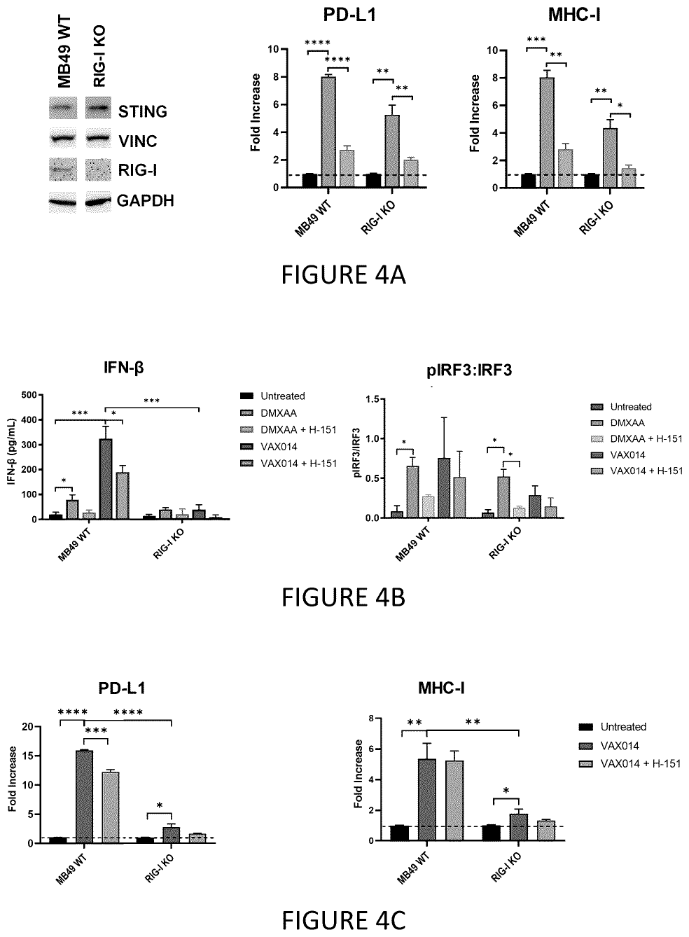

A biallelic targeted deletion of the murine RIG-I gene was generated using a commercially available CRISPR-Cas9 kit to create a MB49-RIG-I KO cell line. Deletion of RIG-I and the preservation of STING expression was confirmed by Western blot (FIG. 4A). Loss of RIG-I function in the MB49-RIG-I KO line was confirmed by a loss of PD-L1 and MHC-I in response to treatment with VAX014 (FIG. 4A). VAX014 still maintained the ability to upregulate IFN-β at a detectable level in the MB49-RIG-I KO cell line (FIG. 4B). Commensurate reductions in the pIRF3/IRF3 ratio (FIG. 4B) along with reduced surface expression of PD-L1 and MHC-I were also observed (FIG. 4C). Preincubation with H-151 lowered each of IFN-β production, pIRF3/IRF3 ratios, and PD-L1/MHC-I surface expression nearly to baseline conditions of the untreated control, thus confirming that VAX014 also activates RIG-I.

Example 7: Genetic Ablation of Both Sting and RIG-I Confirms that Oncolytic Bacterial rBMCs Activate both Pathways

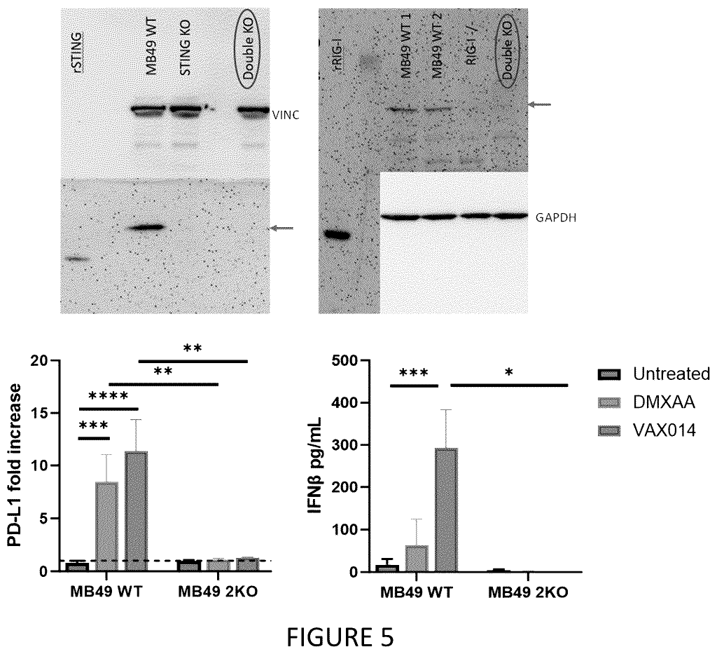

Genetic ablation of both STING and RIG-I in the same model cell line resulted in complete abrogation of Type I IFN (FIG. 5), confirming the dual activation of these pathways by the dual STING/RIG-I oncolytic rBMCs described herein. Biallelic targeted deletion of both the murine STING and RIG-I genes was generated using commercially available CRISPR-Cas9 kit in series to create a MB49-Double KO (MB49 2KO) cell line. Deletion of both STING (top left) and RIG-I (top right) was confirmed by Western blot using Vinculin (VINC) and GAPDH as respective protein loading controls. Complete loss of surface PD-L1 expression (bottom left) and IFN-β production (bottom right) in response to treatment with either DMXAA or VAX014 was observed. Results of these studies further confirm VAX014 activates both the STING and RIG-I pathways to elicit Type I IFN production. As shown in FIGS. 1A-1D, this response is dependent on the presence of the endosomal escape protein, PFO.

### Example 8: Oncolytic Bacterial rBMCs Function in STING Positive and RIG-I Positive Tumors

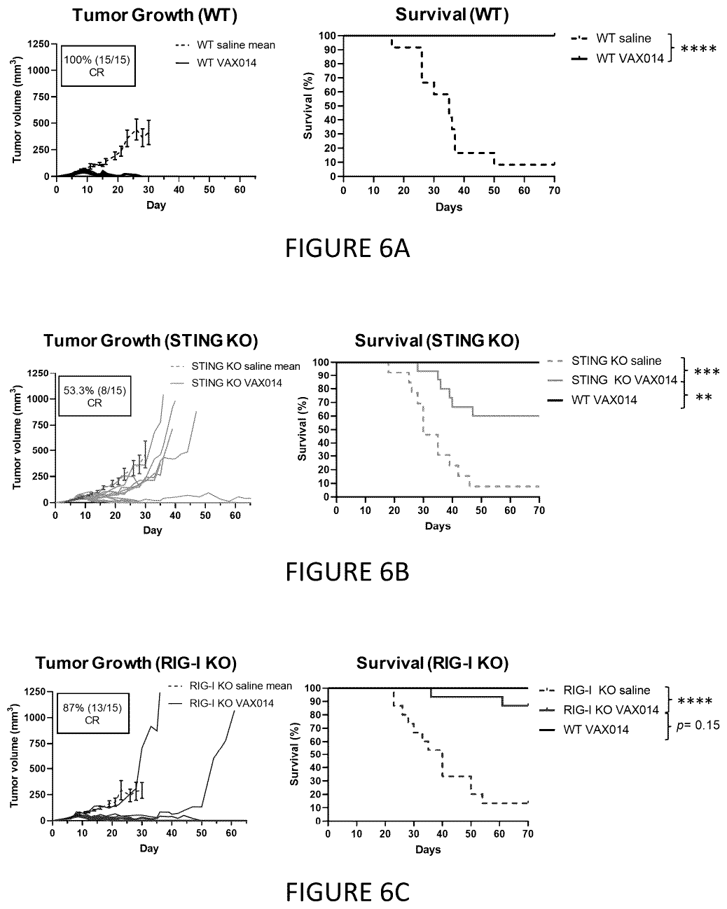

The importance of activating STING and/or RIG-I is demonstrated in vivo (FIG. 6A-6C) where it was found that the presence of both tumor intrinsic STING and RIG-I led to a 100% durable complete response rate compared to a lower complete response and survival rates when tumor intrinsic STING or RIG-I was genetically ablated. Thus, not only are embodiments described herein improved at dual activation of STING and RIG-I, this activity is shown to be critically important to treating tumors that intrinsically express STING and/or RIG-I. This represents an unexpected and major advancement in the field of oncolytic agents, as it is well known that oncolytic viral therapies do not work well and are contraindicated in tumors that express STING and/or RIG-I. Along the same lines, the therapeutic objective of embodiments described herein is to achieve durable tumor specific immunologic memory, a phenomenon commonly referred to as in situ immunization. Wild type female C57BL/6 mice (n=15/group) were inoculated with intradermal WT MB49 tumor cells (FIG. 6A), MB49 STING KO tumor cells (FIG. 6B), or MB49 RIG-I KO tumor cells (FIG. 6C). When tumors approached 50-70 mm³, mice were randomized into respective treatment groups and VAX014 was administered via the intratumoral route weekly for 6 weeks or until tumors regressed to a point where there was no tumor left to inject. Saline (dotted lines) was provided as a control. Individual tumor growth rates of each animal are plotted against the mean tumor growth rate of the saline treated control group (left column). The frequency of complete response (CR) is also enumerated, and the survival curves of each respective group are plotted (right column). Results of these experiments indicate that VAX014 works best in tumors expressing STING and/or RIG-I, providing further evidence that activation of these two pathways by VAX014 is important for activity in vivo.

### Example 9: Antitumor Activity of Oncolytic Bacterial rBMCs is T-Cell Dependent and Leads to Tumor Specific Antitumor Immunologic Memory

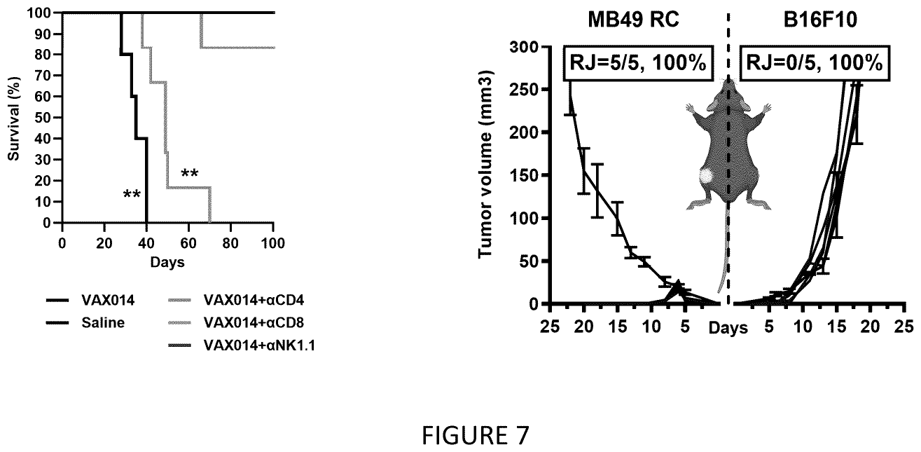

To that end, the antitumor activity of dual STING/RIG-I agonist oncolytic rBMCs was shown to be dependent on T cells (FIG. 7) and resulted in protective tumor specific antitumor immunologic memory (FIG. 7). Female C57BL/6 mice (n=8-10/group) were inoculated with a single intradermal WT MB49 tumor and tumor growth rates monitored until they reached 50-70 mm³ at which time weekly intratumoral administration of VAX014 was initiated (left). Immune cell depletion antibodies (100 μg/dose) were given intraperitoneally starting the day before treatment initiation and continued on a bi-weekly schedule for 5 total administrations. Mice were depleted of CD8+T cells, CD4+T cells, or Natural Killer cells and survival was compared to that of the non-depleted VAX014 treated group. Mice that had achieved a complete response (CR) in response to VAX014 were rechallenged with an MB49 tumor on one flank and a syngeneic non-specific control B16F10 tumor on the opposite flank (right). Results of these studies demonstrate VAX014-mediates in situ immunization.

Another novel and enabling aspect of the present disclosure is improved coordination between the timing between Type I IFN production and tumor antigen release mediated by the oncolytic activity of dual STING/RIG-I agonistic oncolytic rBMCs. To this end, an example provided herein (FIG. 7) demonstrates the rapid production of IFN-β followed by several isoforms of IFN-α (all members of the Type I IFN family) in tumors shortly after intratumoral administration of VAX014 dual STING/RIG-I oncolytic rBMCs in vivo. In addition, the kinetics of oncolysis in the same model tumor cell line demonstrates that peak oncolysis occurs during the peak Type IFN response (FIG. 7). This novel feature ensures the availability of tumor antigens at a time when professional antigen presenting cells, such as conventional dendritic cells (cDCs) are being exposed to high levels of Type I IFN. In this manner, activation of APCs (e.g. cDCs) results in adequate and robust tumor antigen uptake prior to migration to secondary lymphoid tissues where cross-presentation and activation of naïve tumor-specific T cells occurs.

### Example 10: Induction of Type I IFN Production After Intratumoral Administration of Oncolytic Bacterial rBMCs In Vivo Overlaps with Peak Oncolysis

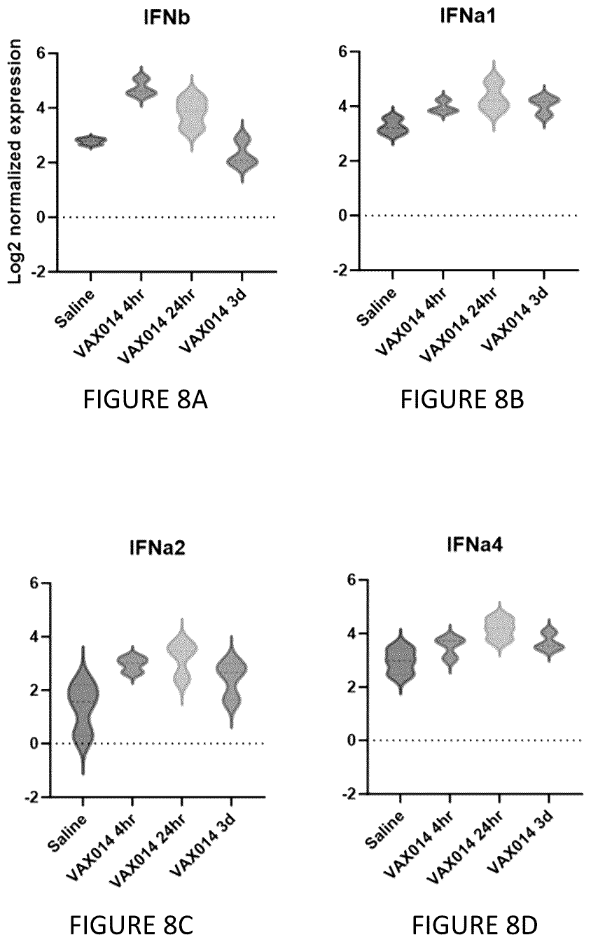

Female C57BL/6 mice (n=3/group/timepoint) were implanted with a single WT MB49 intradermal tumor. When tumors reached 50-70 mm³, a single dose of VAX014 was administered via the intratumoral route. At timepoints of 4 hr, 24 hr, and 3 days, mice were euthanized, tumors were removed, and total RNA was extracted followed by analysis of immunotranscriptomes using NanoString Mouse PanIO-Cancer platform. Individual transcripts for Type I IFNs included in the panel are shown (FIGS. 8A-8D). Corresponding in vitro studies demonstrate that VAX014 facilitates peak oncolysis within 2 hrs as measured by cytosolic LDH release into the cell culture medium. Together, these results demonstrate that dual STING/RIG-I agonistic oncolytic rBMCs produce peak Type I IFN (IFN-B) during a time when peak oncolysis occurs.

Example 11: Oncolytic Bacterial rBMCs Loaded with Exogenous ADU-S100 is PFQ Dependent and More Potent than Free ADU-S100

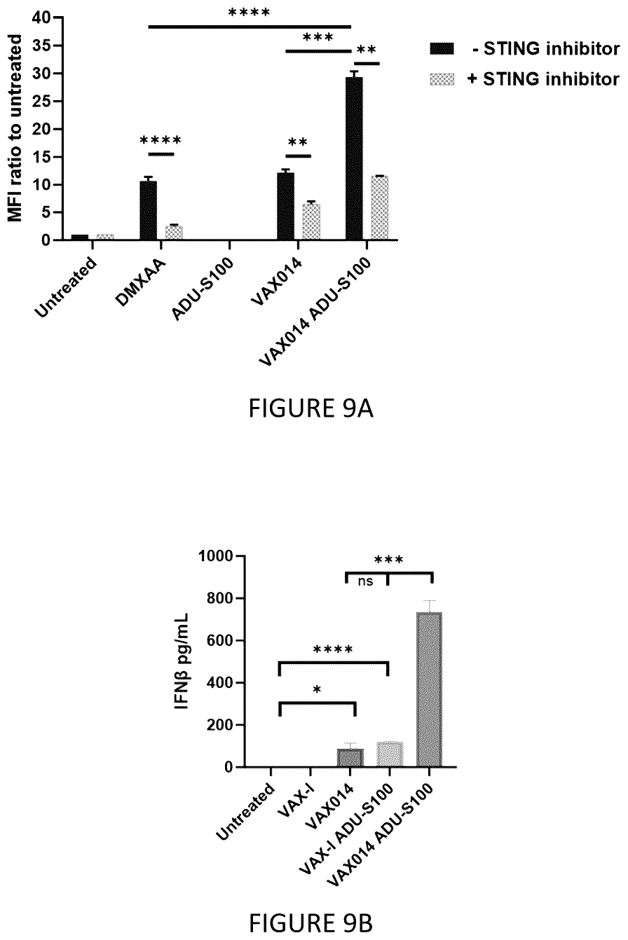

Embodiments of the dual STING/RIG-I oncolytic rBMCs described herein can be further engineered to encapsulate and deliver exogenous agonists of cytosolic nucleic acid receptors that stimulate innate immune responses. The optimal delivery of such agonists is again dependent on the incorporation as an endosomal escape agent (the lack of which is where the prior art falls short). The example provided here (FIGS. 9A-9B) demonstrates that dual STING/RIG-I agonistic oncolytic rBMCs can be loaded with a synthetic cyclic di-nucleotide analog with poor intrinsic cell permeability properties (ADU-S100 in the provided example) and utilized to stimulate high level production of Type I IFN. High level production of Type IFN in this example is again dependent upon an endosomal escape protein (PFO in this case). WT MB49 cells were plated at equal densities (70% confluent) and treated with 10 μg/mL DMXAA (mouse specific STING agonist with excellent cell permeability), 30 μg/mL ADU-S100 (a pan-allelic, pan-species STING agonist with poor cell permeability), VAX014 rBMCs, or an equivalent amount of VAX014 rBMCs that had been loaded with ADU-S100 (FIG. 9A). Each condition contained a corresponding control condition utilizing pretreatment with the irreversible STING inhibitor, H-151 (hashed bars). VAX014 rBMCs were loaded with ADU-S100 by incubating VAX014 rBMCs overnight in 1 mL of 30 μg/mL ADU-S100 at 4° C. VAX014 rBMCs loaded with ADU-S100 were washed by pelleting and resuspension 3X to remove excess ADU-S100 before use in experiments. In this experiment, free ADU-S100 was used at a concentration of 30 μg/mL to represent a condition that assumes all available ADU-S100 had been loaded into VAX014 rBMCs, which condition also controlled for any potential leakage of ADU-S100 from VAX014 rBMCs. The concentration of IFN-β in treated cell culture supernatants was also compared by ELISA from those conditions where no STING inhibitor had been included (FIG. 9B). Results of these studies clearly demonstrate that the endosomal escape protein (PFO in this case) is critical for STING activation by the STING agonist ADU-S100 when delivering ADU-S100 to target cells using rBMCs.

### Example 12: Oncolytic Bacterial rBMCs Mediated PFO-Dependent Activation of Type I IFN Observed in Human Tumor Cell Lines

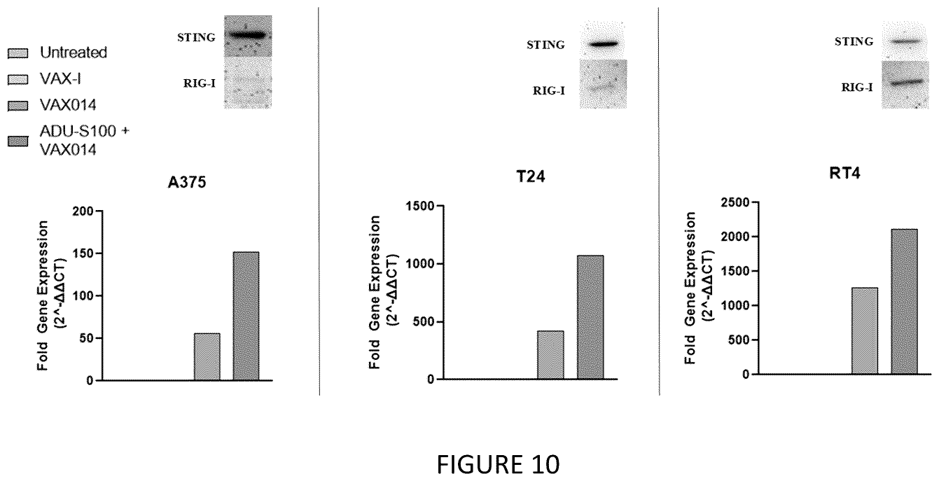

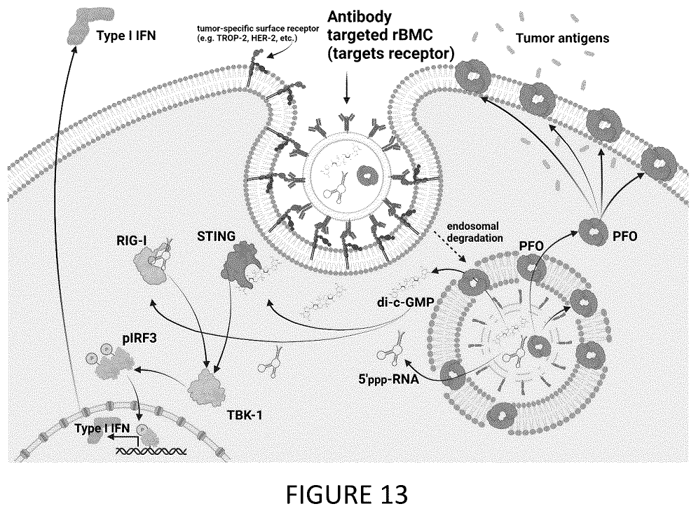

The same results were observed using a panel of human tumor cell lines (FIG. 10), demonstrating a high probability of successful clinical translation into humans. Human tumor cell lines A375 (melanoma), T24 (high grade urothelial carcinoma), and RT4 (low grade urothelial carcinoma) were seeded at 70% confluency and incubated overnight with an equivalent amount of either VAX-I (contains invasin and bacterial di-cyclic nucleotides but no PFO endosome escape protein), VAX014 contains invasin, bacterial di-cyclic nucleotides and PFO endosome escape protein), or VAX014-ADU-S100 loaded minicells (includes invasin, bacterial di-cyclic nucleotides, ADU-S100, and PFO endosome escape protein) at a ratio of minicells to mammalian cells of 300:1. Following incubation, total RNA extraction was performed, followed by RNA quantification, normalization, and conversion to cDNA via reverse transcriptase. cDNA libraries from each condition were then probed for relative human IFN-β gene transcription by real time quantitative PCR using commercially available reagents. Expression levels were normalized to respective GAPDH control transcript levels. The STING and RIG-I status of each cell line was confirmed by Westren blot and as depicted in the respective insets. These results confirm VAX014 mediates PFO-dependent activation of Type I IFN production in STING and RIG-I positive human tumor cell lines, thereby demonstarting the function and utility of this approach for use in humans. In some embodiments, the invasin is replaced with any targeting moiety, selected, in part, because the binding of the minicell-surface displayed antibody targeting moiety, Fc-containing antibody derivatives, and/or Fc-containing fusion/conjugate targeting molecules specific for the antigen induce internalization of the targeted minicell, facilitating intracellular payload delivery. Such targeting moiety can include an antibody, such as any target specific antibody described herein or available and known to those of skill in the art (FIG. 13).

These examples are exemplary, and one of skill in the art can appreciate that modifications, alternatives, or other embodiments, including any of those described herein can be modified to include any combination of features recited herein.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

In addition, where features or aspects of the disclosure are described in terms of Markush groups, those of ordinary skill in the art will recognize that the disclosure is also thereby described in terms of any individual member or subgroup of members of the Markush group.

As will be understood by one ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A recombinant bacterial minicell (rBMC) for the inhibition or treatment of cancer, comprising:
a surface localized targeting molecule;
a cytolysin protein; and
an agonist of an intracellular mediator of a Type I IFN response.

2. The rBMC of claim 1, wherein the surface localized targeting molecule comprises invasin.

3. The rBMC of claim 1, wherein the cytolysin protein comprises perfringolysin O (PFO).

4. The rBMC of claim 1, wherein the agonist is a STING agonist, a RIG-I agonist, or both.

5. The rBMC of claim 4, wherein the STING agonist comprises c-di-GMP, 2′3′cGAMP, 3′3′cGAMP, cAIMP, cAIMP difluoro, cAIMP (PS) 2 Difluor (Rp/Sp), a cyclic di-nucleotide, ADU-S100, MK-1454, BMS-986301, E7766, GSK3745417, SB 11285, or TAK-676, or any combination thereof.

6. The rBMC of claim 4, wherein the RIG-I agonist comprises an uncapped 5′triphosphate RNA.

7. The rBMC of claim 6, wherein the RIG-I agonist comprises polyI:C, SLR14, polyU/UC, RN7SL1, M8, 3p-siBCL2, MK-4621, and BO-112.

8. The rBMC of claim 1, wherein the rBMC expresses one or more recombinant tumor selective antigens.

9. The rBMC of claim 8, wherein the one or more recombinant tumor selective antigens is HER-2, K-RAS, H-RAS, N-RAS, MAGE, c-MYC, MUC-1, PSMA, CEA, ETA, CA-125, p53, AFP, Tyrosinase, oncofetal protein, or antigens produced by oncogenic viruses.

10. A pharmaceutical composition comprising:
a recombinant bacterial minicell as recited in claim 1; and
a pharmaceutically acceptable carrier.

11. The pharmaceutical composition of claim 10, further comprising an immune checkpoint inhibitor.

12. A method of inhibiting or treating cancer, comprising:
administering to a subject having cancer a pharmaceutical composition comprising a targeted oncolytic recombinant bacterial minicell (rBMC),
wherein the bacterial rBMC is configured to stimulate upstream intracellular mediators of a Type I IFN response,
wherein the intracellular mediators of the Type I IFN response comprise stimulator of interferon genes (STING) and retinoic acid inducible gene I (RIG-I) nucleic acid sensing pathways.

13. The method of claim 12, wherein said method inhibits the growth of cancer.

14. The method of claim 12, wherein the cancer is a solid tumor, a metastatic tumor, or a liquid tumor.

15. The method of claim 12, wherein the cancer is epithelial, fibroblast, muscle, or bone origin.

16. The method of claim 12, further comprising administering an immune checkpoint inhibitor therapy.

17. The method of claim 16, wherein the immune checkpoint inhibitor therapy comprises administration of one or more immune checkpoint inhibitors.

18. The method of claim 17, wherein the one or more immune checkpoint inhibitors comprises an inhibitor against PD-1, PD-L1, PD-L2, PD-L3, PD-L4, CTLA-4, LAG-3, IDO, B7-H3, B7-H4, GITR, TIGIT, 4-1BB, OX40, CD27, KIR2DL, CSFIR, CD40L, KIR, or TIM-3.

19. The method of claim 12, wherein the rBMC comprises invasin.

20. The method of claim 12, wherein the rBMC comprises perfringolysin O (PFO).
